(19) United States
(12) Patent Application Publication
(10) Pub. No.: US 2009/0131437 A1
(43) Pub. Date: May 21, 2009

(54) DISUBSTITUTED UREAS AS KINASE INHIBITORS

(75) Inventors: Pascal Furet, Thann (FR); Georg Martiny-Baron, Herbolzheim (DE); Clive McCarthy, Basel (CH); Vittorio Rasetti, Riehen (CH); Andrea Vaupel, Riehen (CH)

Correspondence Address: NOVARTIS;CORPORATE INTELLECTUAL PROPERTY, ONE HEALTH PLAZA 104/3, EAST HANOVER, NJ, 07936-1080, US

(73) Assignees: CORIUM INTERNATIONAL, INC., Redwood City, CA (US); A.V. TOPCHIEV INSTITUTE OF PETROCHEMICAL SYNTHESIS, Moscow (RU)

(21) Appl. No.: 11/813,067

(22) PCT Filed: Jan. 3, 2006
(86) PCT No.: PCT/US2006/000098

§ 371 Date: Nov. 11, 2008

(30) Foreign Application Priority Data
Jan. 10, 2005 (GB) 05004435.3

(51) Int. Cl. A61K 31/5377; C07D 413/14

(52) U.S. Cl. 514/235.8; 544/123

(57) ABSTRACT

The invention relates to compounds of the formula (I), their use as kinase inhibitors, new pharmaceutical formulations comprising said compounds, said compounds for use in the diagnostic or therapeutic treatment of warm-blooded animals, especially humans, their use in the treatment of diseases or for the manufacture of pharmaceutical formulations useful in the treatment of diseases that respond to modulation of kinase activity, methods of treatment comprising administration of said compounds to a warm-blooded animal, especially a human, and processes for the manufacture of said compounds.

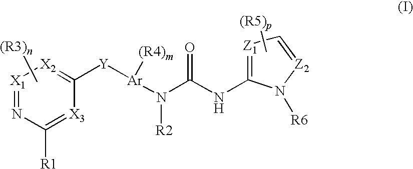

The invention relates to N-(aryl- or heteroaryl)-N′-pyrazinyl urea compounds, their use as kinase inhibitors, new pharmaceutical formulations comprising said compounds, said compounds for use in the diagnostic or therapeutic treatment of warm-blooded animals, especially humans, their use in the treatment of diseases or for the manufacture of pharmaceutical formulations useful in the treatment of diseases that respond to modulation of kinase, especially tie-2 kinase, activity, methods of treatment comprising administration of said compounds to a warm-blooded animal, especially a human, and processes for the manufacture of said compounds.

Among the kinases, receptor-type kinases and nonreceptor-type kinases can be distinguished, as well as tyrosine and serine/threonine kinases. Among the receptor type tyrosine kinases, Tie-2 (which is also called TEK) is expressed in endothelial cells that line the lumen of blood vessels. It has been shown to be involved in endothelial cell migration, sprouting, survival and periendothelic cell recruitment during angiogenesis.

Unlike VEGFRs (vascular endothelial growth factor receptors), which control the onset of angiogenesis, angiopoietins (ligands of Tie-2) and Tie-2 are involved in vessel stabilization and vascular remodeling. It is known that Tie-2 is activated by one of its ligands, angiopoietin-1, which is antagonized by a second ligand, angiopoietin-2 (ang2). In sites where angiogenesis takes place, the antagonist ang2 is up-regulated. Thus it has not yet been possible to reasonably conclude whether inhibition of Tie-2 promotes or inhibits angiogenesis.

On the other hand, in view of the many possible mechanisms involved in the pathogenesis of tumor and other proliferative diseases, a need exists to find novel and useful modulators of the activity of kinases which often are involved in their genesis. If it could, for example, be shown that compounds that modulate Tie-2 activity can affect tumor growth and angiogenesis, this might provide a novel approach to target tumor vessels which are not affected by VEGFR inhibition.

It is therefore a problem to be solved by the present invention to provide novel chemical compounds with advantageous properties that are useful in the treatment of proliferative diseases, such as tumor diseases.

Surprisingly, it is possible to establish that a novel class of N-(aryl- or heteroaryl)-N′-pyrazinyl urea compounds is capable to inhibit the growth of tumors in tumor models that depend on angiogenesis. Especially, it has been found that these compounds can inhibit Tie-2 kinase quite specifically and are sufficient to inhibit VEGF-induced angiogenesis in vivo when tested, for example, in a subcutaneous growth factor chamber implant model and can show, for example, qualitative differences to VEGFR2 inhibitors.

The present invention relates especially to compounds of the formula I,

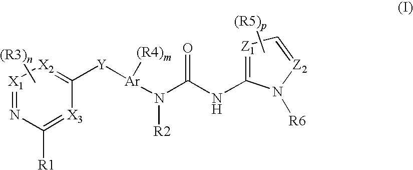

wherein
R1 is hydrogen, or unsubstituted or substituted alkyl, halogen, hydroxy, esterified or etherified hydroxy, amino, substituted amino, carboxy, esterified carboxy, carbamoyl, N-mono- or N,N-disubstituted carbamoyl;
R2 is unsubstituted or substituted alkyl, unsubstituted or substituted aryl, unsubstituted or substituted heterocyclyl or unsubstituted or substituted cycloalkyl,
n is 0, 1, 2 or 3;
m is 0, 1, 2 or 3;
p is 0, 1, 2 or 3;
each of R3 and R4, if present and independently of the others, is unsubstituted or substituted alkyl, halogen, hydroxy, esterified or etherified hydroxy, mercapto, substituted mercapto, nitro, amino, substituted amino, carboxy, esterified carboxy, carbamoyl, N-mono- or N,N-disubstituted carbamoyl, sulfo, esterified sulfo, sulfamoyl, N-mono- or N,N-disubstituted sulfamoyl or cyano;
R5, independently of R3 and R4, is unsubstituted or substituted alkyl, unsubstituted or substituted aryl, unsubstituted or substituted heterocyclyl or unsubstituted or substituted cycloalkyl, halogen, hydroxy, esterified or etherified hydroxy, mercapto, substituted mercapto, nitro, amino, substituted amino, carboxy, esterified carboxy, carbamoyl, N-mono- or N,N-disubstituted carbamoyl, sulfo, esterified sulfo, sulfamoyl, N-mono- or N,N-disubstituted sulfamoyl or cyano;
R6 is unsubstituted or substituted alkyl, unsubstituted or substituted aryl, unsubstituted or substituted heterocyclyl or unsubstituted or substituted cycloalkyl;
each of X₁, X₂ and X₃, independently of the others, is N or CH;
Y is oxy (—O—), imino (—NH—), thio (—S—) or methylene (—CH₂—) and
Ar is arylene or heterocyclylene;
and each of Z₁ and Z₂, independently of the other, is nitrogen (N) or CH, with the proviso that
at least one of Z₁ and Z₂ is N;
or (preferably pharmaceutically acceptable) salts thereof.

Listed below are definitions of various terms used to describe the compounds of the present invention as well as their use and synthesis, starting materials and intermediates and the like. These definitions, either by replacing one, more than one or all general expressions or symbols used in the present disclosure and thus yielding preferred embodiments of the invention, preferably apply to the terms as they are used throughout the specification unless they are otherwise limited in specific instances either individually or as part of a larger group. In other terms: Independently of each other, one or more of the more general expressions may be replaced by the more specific definitions, thus leading to preferred embodiments of the invention.

The term “lower” or “C₁-C₇-” defines a moiety with up to and including maximally 7, especially up to and including maximally 4, carbon atoms, said moiety being branched (one or more times) or straight-chained and bound via a terminal or a non-terminal carbon. Lower or C₁-C₇-alkyl, for example, is n-pentyl, n-hexyl or n-heptyl or preferably C₁-C₄-alkyl, especially as methyl, ethyl, n-propyl, sec-propyl, n-butyl, isobutyl, sec-butyl, tert-butyl.

Halo or halogen is preferably fluoro, chloro, bromo or iodo, most preferably fluoro, chloro or bromo.

Unsubstituted or substituted alkyl is preferably C₁-C₂₀-alkyl, more preferably C₁-C₇-alkyl, that is straight-chained or branched (one or, if desired and possible, more times), and which is unsubstituted or substituted by one or more, e.g. up to three moieties selected from unsubstituted or substituted aryl as described below, especially phenyl or naphthyl, (each of) which is unsubstituted or substituted as described below for unsubstituted or substituted aryl, unsubstituted or substituted heterocyclyl as described below which is unsubstituted or substituted as described below for unsubstituted or substituted heterocyclyl, especially piperidino, morpholino, thiomorpholino, N—C₁-C₇-alkyl-piperazino, or N-mono- or N,N-di-(C₁-C₇-alkyl-substituted or unsubstituted pyrrolidino, unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, e.g. in trifluoromethyl, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl and/or C₁-C₇-alkoxy-C₁-C₇-alkyl and/or mono- or di-(C₁-C₇-alkyl)-amino-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkynylene, C₁-C₇-alkylenedioxy, sulfeno, O—C₁-C₇-alkylsulfenyl, O-phenyl- or naphthylsulfenyl wherein phenyl or naphthyl is unsubstituted or substituted by one or more, especially one to three, C₁-C₇-alkyl moieties, O-phenyl- or naphthyl-C₁-C₇-alkylsulfenyl, sulfino, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfo, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl.

Unsubstituted or substituted aryl preferably is a mono- or polycyclic, especially monocyclic, bicyclic or tricyclic aryl moiety with 6 to 22 ring carbon atoms, especially phenyl (very preferred), naphthyl (very preferred), indenyl, fluorenyl, acenapthylenyl, phenylenyl or phenanthryl, and is unsubstituted or substituted by one or more, especially one to three, moieties, preferably independently selected from the group consisting of C₁-C₇-alkyl, such as methyl, ethyl, n-propyl, isopropyl, n-butyl, isobutyl, sec-butyl or tert-butyl, C₂-C₇-alkenyl, C₂-C₇-alkinyl, phenyl- or naphthyl-C₁-C₇-alkyl, such as benzyl or naphthylmethyl, halo-C₁-C₇-alkyl, such as trifluoromethyl, hydroxy-C₁-C₇-alkyl, C₁-C₇-alkoxy-C₇-C₇-alkyl, such as 3-methoxypropyl or 2-methoxyethyl, C₁-C₇-alkoxy-C₁-C₇-alkoxy-C₁-C₇-alkyl, phenyloxy- or naphthyloxy-C₁-C₇-alkyl, phenyl-C₁-C₇-alkoxy- or naphthyl-C₁-C₇-alkoxy-C₁-C₇-alkyl, amino-C₁-C₇-alkyl, such as aminomethyl, N-mono- or N,N-di-(C₁-C₇-alkyl and/or mono-C₁-C₇-alkoxy-C₁-C₇-alkyl and/or (mono- or di-(C₁-C₇-alkyl)-amino-C₁-C₇-alkyl)amino-C₁-C₇-alkyl, C₁-C₇-alkoxy-C₁-C₇-alkylamino-C₁-C₇-alkyl, mono- or di-(naphthyl- or phenyl-C₁-C₇-alkyl)-amino-C₁-C₇-alkyl, C₁-C₇-alkanoyl-amino-C₁-C₇-alkyl, carboxy-C₁-C₇-alkyl, benzoyl- or naphthoylamino-C₁-C₇-alkyl, C₁-C₇-alkylsulfonylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkylpiperazino-C₁-C₇-alkyl, N-mono- or N,N-di-(C₁-C₇-alkyl)-amino-substituted or unsubstituted pyrrolidino-C₁-C₇-alkyl, halo, especially fluoro, chloro or bromo, hydroxy, C₁-C₇-alkoxy, phenyl-C₁-C₇-alkoxy wherein phenyl is unsubstituted or substituted by C₁-C₇-alkoxy and/or halo, halo-C₁-C₇-alkoxy, such as trifluoromethoxy, hydroxy-C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, N-unsubstituted-, N-mono- or N,N-di-(C₁-C₇-alkyl)carbamoyl-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, C₁-C₇-alkanoyl, C₁-C₇-alkoxy-C₁-C₇-alkanoyl, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl and/or mono-C₁-C₇-alkoxy-C₁-C₇alkyl and/or (mono- or di-(C₁-C₇-alkyl)-amino-C₁-C₇-alkyl)amino-carbonyl, such as N-mono- or N,N-di-(C₁-C₇-alkyl)-aminocarbonyl, N—C₁-C₇-alkoxy-C₁-C₇-alkylcarbamoyl, N-mono- or N,N-di-(naphthyl- or phenyl-C₁-C₇-alkyl)-aminocarbonyl, pyrrolidinocarbonyl, piperidinocarbonyl, morpholinocarbonyl, thiomorpholinocarbonyl, N—C₁-C₇-alkyl-piperazinocarbonyl, N-mono- or N,N-di-(C₁-C₇-alkyl)-amino-substituted or unsubstituted pyrrolidino-C₁-C₇-alkyl, cyano, C₁-C₇-alkenylene or -alkinylene, C₁-C₇-alkylenedioxy, sulfeno, O—C₁-C₇-alkylsulfenyl, O-phenyl- or naphthylsulfenyl wherein phenyl or naphthyl is unsubstituted or substituted by one or more, especially one to three, C₁-C₇-alkyl moieties, O-phenyl- or naphthyl-C₁-C₇-alkylsulfenyl, sulfino, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfo, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl-, phenyl-C₁-C₇-alkyl- or naphthyl-C₁-C₇-alkyl)-aminosulfonyl, piperidino, morpholino, thiomorpholino, N—C₁-C₇-alkyl-piperazino, or N-mono- or N,N-di-(C₁-C₇-alkyl)amino-substituted or unsubstituted pyrrolidino. Especially preferably aryl is phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, halo-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, N-mono- or N,N-di-(C₁-C₇-alkyl)-amino-substituted or unsubstituted pyrrolidino-C₁-C₇-alkyl, halo, especially fluoro, chloro or bromo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N-mono- or N,N-di-(C₁-C₇-alkyl)carbamoyl-C₁-C₇-alkoxy, amino, C₁-C₇-alkanoylamino, C₁-C₇-alkanoyl, C₁-C₇ alkoxy-C₁-C₇-alkanoyl, carboxy, C₁-C₇-alkoxycarbonyl, carbamoyl, N-mono- or N,N-di-(C₁-C₇ alkyl and/or C₁-C₇-alkoxy-C₁-C₇-alkyl)-carbamoyl, pyrrolidinocarbonyl, piperidinocarbonyl, morpholinocarbonyl, thiomorpholinocarbonyl, N—C₁-C₇-alkyl-piperazinocarbonyl, N-mono- or N,N-di-(C₁-C₇-alkyl)-amino-substituted or unsubstituted pyrrolidino-C₁-C₇-alkyl, nitro, cyano, pyrrolidino, piperidino, morpholino, thiomorpholino, N—C₁-C₇-alkyl-piperazino, and N-mono- or N,N-di-(C₁-C₇-alkyl)-amino-substituted or unsubstituted pyrrolidino.

Where unsubstituted or substituted heterocyclyl is mentioned, heterocyclyl is preferably a heterocyclic radical that is unsaturated, saturated or partially saturated in the bonding ring and is preferably a monocyclic or in a broader aspect of the invention poly-, e.g. bi- or tri-cyclic ring; has 3 to 24, more preferably 4 to 16 ring atoms; wherein at least in the ring bonding to the remaining part of the molecule of formula I one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; heterocyclyl being unsubstituted or substituted by one or more, especially 1 to 3, substituents independently selected from the group consisting of the substituents defined above under “substituted alkyl” or “substituted aryl”; especially being a heterocyclyl radical selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals selected from the group consisting of lower alkyl, especially methyl or tert-butyl, lower alkoxy, especially methoxy, and halo, especially bromo or chloro.

Unsubstituted or substituted cycloalkyl is preferably mono- or polycyclic, more preferably monocyclic, C₃-C₁₆-cycloalkyl, especially C₃-C₁₀-cycloalkyl which may include one or more double (e.g. in cycloalkenyl) and/or triple bonds (e.g. in cycloalkynyl), and is unsubstituted or substituted by one or more, e.g. one to three substituents preferably independently selected from those mentioned above as substituents for substituted alkyl or substituted aryl. Preferred is cyclopropyl, cyclobutyl, cyclopentyl, cyclohexyl or cycloheptyl.

n is 0, 1, 2 or 3, preferably 0 or 1, e.g. 0.
m is 0, 1, 2 or 3, preferably 0 or 1, e.g. 0.
p is 0, 1, 2 or 3, preferably 1 or 2, most preferably 1.

R5 is most preferably present at position 5 in the pyrazol ring in formula I (p=1) and is preferably substituted or most preferably unsubstituted alkyl, especially branched C₁-C₇-alkyl.

Esterified or etherified hydroxy is preferably hydroxy etherified by unsubstituted or substituted lower alkyl which is preferably as defined above, and is more preferably lower-alkoxy, (lower-alkoxy)-lower alkoxy, phenoxy, naphthoxy, phenyl-lower alkoxy, such as benzyloxy, or naphthyl-lower alkoxy; or hydroxy esterified by an organic carbonic or sulfonic acid, e.g. lower alkanoyloxy, lower alkoxy-carbonyloxy, such as tert-butoxycarbonyloxy, phenyl-lower alkoxy-carbonyloxy, such as benzyloxycarbonyloxy, methylphenylsulfonyloxy or lower-alkylsulfonyloxy.

Substituted mercapto can be mercapto that is thioesterified with acyl as defined below, especially with lower alkanoyloxy; or preferably thioetherified with alkyl, aryl, heterocyclyl or cycloalkyl each of which is unsubstituted or substituted and is preferably as described above for the corresponding unsubstituted or substituted moieties. Especially preferred is unsubstituted or especially substituted C₁-C₇-alkylthio or unsubstituted or substituted arylthio with unsubstituted or substituted C₁-C₇-alkyl or aryl as just described for the corresponding moieties under etherified hydroxy.

Acyl is preferably unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl or unsubstituted or substituted alkylcarbonyl or -sulfonyl, wherein unsubstituted or substituted aryl, unsubstituted or substituted heterocyclyl, unsubstituted or substituted cycloalkyl and unsubstituted or substituted alkyl are preferably as described above. Preferred is C₁-C7-alkanoyl, C₁-C₇-alkylsulfonyl or (unsubstituted or C₁-C₇-alkyl-substituted) phenylsulfonyl.

Substituted amino can be mono- or di-substituted amino, wherein amino is preferably substituted by one or two substituents selected from one acyl, especially C₁-C₇-alkanoyl, C₁-C₇-alkylsulfonyl or phenylsulfonyl wherein phenyl is unsubstituted or substituted by one to 3 C₁-C₇-alkyl groups, and one or two moieties selected from alkyl, aryl, heterocyclyl and cycloalkyl each of which is unsubstituted or substituted and is preferably as described above for the corresponding unsubstituted or substituted moieties. Preferred is C₁-C₇-alkanoylamino, mono- or di-[C₁-C₇-alkyl and/or C₁-C₇-alkoxy-C₁-C₇-alkyl and/or (mono- or di-(C₁-C₇-alkyl)-amino-C₁-C₇-alkyl]-amino or mono- or di-(naphthyl- or phenyl-C₁-C₇-alkyl)-amino, or (where the binding nitrogen forms part of a ring) piperidino, morpholino, thiomorpholino, N—C₁-C₇-alkylpiperazino, or N-mono- or N,N-di-(C₁-C₇-alkyl-substituted or unsubstituted pyrrolidino.

Esterified carboxy is preferably alkyloxycarbonyl, aryloxycarbonyl, heterocyclyloxycarbonyl or cycloalkyloxycarbonyl, wherein alkyl, aryl, heterocyclyl and cycloalkyl are unsubstituted or substituted and the corresponding moieties and their substituents are preferably as described above. Preferred is C₁-C₇-alkoxycarbonyl, phenyl-C₁-C₇-alkoxycarbonyl, phenoxycarbonyl or naphthoxycarbonyl.

In (N—)mono- or (N,N-)disubstituted carbamoyl (=amidated carboxy), the amino part is unsubstituted or substituted as described for substituted amino, but preferably without acyl as amino substituent, or is an N that forms part of an unsubstituted or substituted heterocyclyl ring, especially, pyrrolidino, morpholino, thiomorpholino, piperazino or N—C₁-C₇-alkylpiperazino. Preferred is mono- or di-(C₁-C₇-alkyl and/or C₁-C₇-alkoxy-C₁-C₇-alkyl)-aminocarbonyl, mono- or di-(naphthyl- or phenyl-C₁-C₇-alkyl)aminocarbonyl, pyrrolidinocarbonyl, morpholino-carbonyl, thiomorpholinocarbonyl, pyrrolidinocarbonyl, piperazinocarbonyl or N—C₁-C₇-alkylpiperazinocarbonyl.

Esterified sulfo is preferably alkyloxysulfonyl, aryloxysulfonyl, heterocyclyloxysulfonyl or cycloalkyloxysulfonyl, wherein alkyl, aryl, heterocyclyl and cycloalkyl are unsubstituted or substituted and the corresponding moieties and their substituents are preferably as described above. Preferred is C₁-C₇-alkoxysulfonyl, phenyl-C₁-C₇-alkoxysulfonyl, phenoxysulfonyl or naphthoxysulfonyl.

In N-mono- or N,N-disubstituted sulfamoyl (=amidated sulfono), the amino part is unsubstituted or substituted as described for substituted amino, but preferably without acyl as amino substituent. Preferred is mono- or di-(C₁-C₇-alkyl and/or C₁-C₇-alkoxy-C₁-C₇-alkyl)-aminosulfonyl or mono- or di-(naphthyl- or phenyl-C₁-C₇-alkyl)-aminosulfonyl.

Each of X₁, X₂ and X₃, independently of the others, is N or CH; preferably, X₁ is CH, X₂ is CH or preferably N and X₃ is CH.

Arylene is a bivalent aryl with an aryl ring system as defined above for aryl. Heterocyclylene is a bivalent heterocyclyl with a heterocyclyl ring system as defined above for heterocyclyl.

Preferably either Z₁ is N and Z₂ is CH, or more preferably Z₁ is CH and Z₂ is N.

Salts are especially the pharmaceutically acceptable salts of compounds of formula I. They can be formed where salt forming groups, such as basic or acidic groups, are present that can exist in dissociated form at least partially, e.g. in a pH range from 4 to 10 in aqueous environment, or can be isolated especially in solid form.

Such salts are formed, for example, as acid addition salts, preferably with organic or inorganic acids, from compounds of formula I with a basic nitrogen atom, especially the pharmaceutically acceptable salts. Suitable inorganic acids are, for example, halogen acids, such as hydrochloric acid, sulfuric acid, or phosphoric acid. Suitable organic acids are, for example, carboxylic, phosphonic, sulfonic or sulfamic acids, for example acetic acid, propionic acid, lactic acid, fumaric acid, succinic acid, citric acid, amino acids, such as glutamic acid or aspartic acid, maleic acid, hydroxymaleic acid, methylmaleic acid, benzoic acid, methane- or ethane-sulfonic acid, ethane-1,2-disulfonic acid, benzenesulfonic acid, 2-naphthalenesulfonic acid, 1,5-naphthalene-disulfonic acid, N-cyclohexylsulfamic acid, N-methyl-, N-ethyl- or N-propyl-sulfamic acid, or other organic protonic acids, such as ascorbic acid.

In the presence of negatively charged radicals, such as carboxy or sulfo, salts may also be formed with bases, e.g. metal or ammonium salts, such as alkali metal or alkaline earth metal salts, for example sodium, potassium, magnesium or calcium salts, or ammonium salts with ammonia or suitable organic amines, such as tertiary monoamines, for example triethylamine or tri(2-hydroxyethyl)amine, or heterocyclic bases, for example N-ethyl-piperidine or N,N′-dimethylpiperazine.

When a basic group and an acid group are present in the same molecule, a compound of formula I may also form internal salts.

For isolation or purification purposes it is also possible to use pharmaceutically unacceptable salts, for example picrates or perchlorates. For therapeutic use, only pharmaceutically acceptable salts or free compounds are employed (where applicable comprised in pharmaceutical preparations), and these are therefore preferred.

In view of the close relationship between the compounds in free form and in the form of their salts, including those salts that can be used as intermediates, for example in the purification or identification of the compounds or salts thereof, any reference to “compounds” (including also starting materials and “intermediates”) hereinbefore and hereinafter, especially to the compound(s) of the formula I, is to be understood as referring also to one or more salts thereof or a mixture of a free compound and one or more salts thereof, each of which is intended to include also any solvate, metabolic precursor such as ester or amide of the compound of formula I, or salt of any one or more of these, as appropriate and expedient and if not explicitly mentioned otherwise. Different crystal forms may be obtainable and then are also included.

Where the plural form is used for compounds, salts, pharmaceutical preparations, diseases, disorders and the like, this is intended to mean also a single compound, salt, pharmaceutical preparation, disease or the like, and vice versa.

In some cases, a compound of the present invention comprises one or more chiral centers or show other asymmetry (leading to enantiomers) or may otherwise be able to exist in the form of more than one stereoisomer, e.g. due more than one chiral centers or more than one asymmetry or due to rings or double bonds that allow for Z/E (or cis-trans) isomerism (diastereomers). The present inventions includes both mixtures of two or more such isomers, such as mixtures of enantiomers, especially racemates, as well as preferably purified isomers, especially purified enantiomers or enantiomerically enriched mixtures.

The compounds of formula I have valuable pharmacological properties and are useful in the treatment of kinase, especially Tie-2, dependent diseases, e.g., as drugs to treat one or more proliferative diseases.

The terms “treatment” or “therapy” (especially of tyrosine protein kinase dependent diseases or disorders) refer to the prophylactic or preferably therapeutic (including but not limited to palliative, curing, symptom-alleviating, symptom-reducing, kinase-regulating and/or kinase-inhibiting) treatment of said diseases, especially of the diseases mentioned below.

A warm-blooded animal (or patient) is preferably a mammal, especially a human.

Where subsequently or above the term “use” is mentioned (as verb or noun) (relating to the use of a compound of the formula I or a pharmaceutically acceptable salt thereof), this (if not indicated differently or suggested differently by the context) includes any one or more of the following embodiments of the invention, respectively (if not stated otherwise): the use in the treatment of a protein (especially tyrosine, more especially Tie-2) kinase dependent disease, the use for the manufacture of pharmaceutical compositions for use in the treatment of a protein kinase dependent disease, methods of use of one or more compounds of the formula I in the treatment of a protein kinase dependent and/or proliferative disease, pharmaceutical preparations comprising one or more compounds of the formula I for the treatment of said protein kinase dependent disease, and one or more compounds of the formula I in the treatment of said protein kinase dependent disease, as appropriate and expedient, if not stated otherwise. In particular, diseases to be treated and are thus preferred for “use” of a compound of formula I are selected from (especially tyrosine) protein kinase dependent (“dependent” meaning also “supported”, not only “solely dependent”) diseases mentioned below, especially proliferative diseases mentioned below, more especially any one or more of these or other diseases that depend on Tie-2, e.g. aberrantly highly-expressed, constitutively activated, normal and/or mutated Tie-2 kinase.

The (especially important and preferred) efficacy of compounds of the formula I as inhibitors or Tie-2 kinase can be demonstrated as follows:

### Tie-2 Receptor Autophosphorylation

The inhibition of Tie-2 receptor autophosphorylation can be confirmed with an in vitro experiment in cells such as transfected COS cells (ATCC Number: CRL-1651), which permanently express human Tie-2 (SwissProt AccNo Q02763), are seeded in complete culture medium. (with 10% fetal calf serum=FCS) in 6-well cell-culture plates and incubated at 37° C. under 5% CO₂ until they show about 90% confluency. The compounds to be tested are then diluted in culture medium (without FCS, with 0.1% bovine serum albumin) and added to the cells. Controls comprise medium without test compounds. After 40 min of incubation at 37° C., ortho vanadate is added to give the final concentration of 10 mM. After a further incubation for 20 minutes at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C.

A sandwich ELISA is carried out to measure the Tie-2 phosphorylation: a monoclonal anti-body to Tie-2 (for example anti-Tie2 clone AB33, Upstate, Cat Nr. 05-584 or comparable monoclonal antibody) is immobilized using 0.1 ml of a 2 kg/ml solution on black ELISA plates (OptiPlate™ HTRF-96 from Packard). The plates are then washed and the remaining free protein-binding sites are saturated with 3% TopBlock® (Juro, Cat. #TB232010) in phosphate buffered saline with Tween 20® (polyoxyethylene(20)sorbitane monolaurate, ICI/Uniquema) (PBST). The cell lysates (100 μg protein per well) are then incubated in these plates overnight at 4° C. together with an antiphosphotyrosine antibody coupled with alkaline phosphatase (PY20:AP from Zymed). The (plates are washed again and the) binding of the anti-phosphotyrosine antibody to the captured phosphorylated receptor is then demonstrated using a luminescent AP substrate (CDP-Star, ready to use, with Emerald II; Applied Bio-systems). The luminescence is measured in a Packard Top Count Microplate Scintillation Counter. The difference between the signal of the positive control (stimulated with vanadate) and that of the negative control (not stimulated) corresponds to maximum Tie-2 phosphorylation (=100%). The activity of the tested substances is calculated as percent inhibition of maximum Tie-2 phosphorylation, and the concentration of substance that induces half the maximum inhibition is defined as the IC₅₀ (inhibitory dose for 50% inhibition). For compounds of the formula I, preferably IC₅₀ values in the range from 0.0005 to 5 μM can be found, e.g. more preferably from 0.001 to 1 μM.

### KDR Autophosphorylation

The activity of the compounds of the invention as inhibitors of KDR protein-tyrosine kinase activity can be demonstrated as follows: The inhibition of VEGF-induced receptor autophosphorylation can be confirmed in cells such as transfected CHO cells, which permanently express human VEGF-R2 receptor (KDR), and are seeded in complete culture medium (with 10% fetal calf serum ═FCS) in 6-well cell-culture plates and incubated at 37° C. under 5% CO₂ until they show about 80% confluency. The compounds to be tested are then diluted in culture medium (without FCS, with 0.1% bovine serum albumin) and added to the cells. Controls comprise medium without test compounds. After 2 h incubation at 37° C., recombinant VEGF is added; the final VEGF concentration is 20 ng/ml. After a further incubation period of five minutes at 37° C., the cells are washed twice with ice-cold PBS (phosphate-buffered saline) and immediately lysed in 100 μl lysis buffer per well. The lysates are then centrifuged to remove the cell nuclei, and the protein concentrations of the supernatants are determined using a commercial protein assay (BIORAD). The lysates can then either be immediately used or, if necessary, stored at −20° C.

A good selectivity can also be found using in vitro assays known in the art for CDK1; B-RAF, and IGF.

The results indicate an advantageous selectivity profile of compounds of the formula I with a quite specific inhibition for Tie-2 kinase, where selectivity does not necessarily mean that only Tie-2 kinase is inhibited to an advantageous and pharmaceutically relevant extent.

The efficiency of the compounds of the formula I as inhibitors of tumor growth can be demonstrated as follows:

For example, in order to test whether a compound of the formula I, e.g. that of Example 1 given below, inhibits VEGF-mediated angiogenesis in vivo, its effect on the angiogenic response induced by VEGF in a growth factor implant model in mice is tested: A porous Teflon chamber (volume 0.5 mL) is filled with 0.8% w/v agar containing heparin (20 units/ml) with or without growth factor (2 μg/ml human VEGF) is implanted subcutaneously on the dorsal flank of C57/C6 mice. The mice are treated with the test compound (e.g. 25, 50 or 100 mg/kg p.o. once daily) or vehicle starting on the day of implantation of the chamber and continuing for 4 days after. At the end of the treatment, the mice are killed, and the chambers are removed. The vascularized tissue growing around the chamber is carefully removed and weighed, and the blood content is assessed by measuring the hemoglobin content of the tissue (Drabkins method; Sigma, Deisenhofen, Germany). It has been shown previously that these growth factors induce dose-dependent increases in weight and blood content of this tissue growing (characterized histologically to contain fibroblasts and small blood vessels) around the chambers and that this response is blocked by antibodies that specifically neutralize VEGF (see Wood J M et al., Cancer Res. 60(8), 2178-2189, (2000); and Schlaeppi et al., J. Cancer Res. Clin. Oncol. 125, 336-342, (1999)). With this model, inhibition can be shown in the case of compounds of the formula I e.g. in case of dosages applied in the range from 10 to 100 mg/kg and day in case of oral administration (p.o.).

In view of the high expression of the Tie-2 antagonist angiopoietin-2 expression of which is up-regulated at sites where angiogenesis takes place, this result is surprising. In addition, although VEGF has been used to stimulate angiogenesis in the in vivo model, selective Tie-2 inhibitors are sufficient to inhibit angiogenesis. Thus far it was not clear whether VEGF-driven angiogenesis can be only inhibited by specific inhibitors blocking endothelial receptors other than VEGF receptor.

In a preferred sense of the invention, a disease or disorder dependent on activity of a protein (preferably tyrosine) kinase, especially Tie-2, where a compound of the formula I can be used is one or more of a proliferative disease (meaning one dependent on inadequate including a hyperproliferative condition, such as one or more of leukemia, hyperplasia, fibrosis (especially pulmonary, but also other types of fibrosis, such as renal fibrosis), angiogenesis, psoriasis, atherosclerosis and smooth muscle proliferation in the blood vessels, such as stenosis or restenosis following angioplasty. Further, a compound of the formula I may be used for the treatment of thrombosis and/or scleroderma.

Preferred is the use of a compound of the formula I in the therapy (including prophylaxis) of a proliferative disorder (especially which is dependent on (for example inadequate) Tie-2 activity) selected from tumor or cancer diseases, especially against preferably a benign or especially malignant tumor or cancer disease, more preferably solid tumors, e.g. carcinoma of the brain, kidney, liver, adrenal gland, bladder, breast, stomach (especially gastric tumors), ovaries, colon, rectum, prostate, pancreas, lung (e.g. small or large cell lung carcinomas), vagina, thyroid, sarcoma, glioblastomas, multiple myeloma or gastrointestinal cancer, especially colon carcinoma or colorectal adenoma, or a tumor of the neck and head, e.g. squameous carcinoma of the head and neck, including neoplasias, especially of epithelial character, e.g. in the case of mammary carcinoma; an epidermal hyperproliferation (other than cancer), especially psoriasis; prostate hyperplasia; or a leukemia.

A compound of formula I or its use makes it possible to bring about the regression of tumors and to prevent the formation of tumor metastases and the growth of (also micro)metastases.

Angiogenesis is regarded as an absolute prerequisite for those tumors which grow beyond a maximum diameter of about 1-2 mm; up to this limit, oxygen and nutrients may be supplied to the tumor cells by diffusion. Every tumor, regardless of its origin and its cause, is thus dependent on angiogenesis for its growth after it has reached a certain size. Three principal mechanisms play an important role in the activity of angiogenesis inhibitors against tumors: 1) Inhibition of the growth of vessels, especially capillaries, into avascular resting tumors, with the result that there is no net tumor growth owing to the balance that is achieved between apoptosis and proliferation; 2) Prevention of the migration of tumor cells owing to the absence of blood flow to and from tumors; and 3) Inhibition of endothelial cell proliferation, thus avoiding the paracrine growth-stimulating effect exerted on the surrounding tissue by the endothelial cells normally lining the vessels.

Compounds of the formula I, in regard of their ability to inhibit Tie-2 kinase, and thus to modulate angiogenesis, are especially appropriate for the use against diseases or disorders related to the inadequate activity of Tie-2 kinase, especially an overexpression thereof. Among these diseases, especially e.g. ischemic) retinopathies, (e.g. age related) macula degeneration, psoriasis, obesity, haemangioblastoma, haemangioma, inflammatory diseases, such as rheumatoid or rheumatic inflammatory diseases, especially arthritis, such as rheumatoid arthritis, or other chronic inflammatory disorders, such as chronic asthma, arterial or post-transplantational atherosclerosis, endometriosis, and especially neoplastic diseases, for example so-called solid tumors (especially cancers of the gastrointestinal tract, the pancreas, breast, stomach, cervix, bladder, kidney, prostate, ovaries, endometrium, lung, brain, melanoma, Kaposi's sarcoma, squamous cell carcinoma of head and neck, malignant pleural mesotherioma, lymphoma or multiple myeloma) and further liquid tumors (e.g. leukemias) are especially important.

The compounds of the formula I are especially of use to prevent or treat diseases that are triggered by persistent angiogenesis, such as restenosis, e.g., stent-induced restenosis; Crohn's disease; Hodgkin's disease; eye diseases, such as diabetic retinopathy and neovascular glaucoma; renal diseases, such as glomerulonephritis; diabetic nephropathy; inflammatory bowel disease; malignant nephrosclerosis; thrombotic microangiopathic syndromes; (e.g. chronic) transplant rejections and glomerulopathy; fibrotic diseases, such as cirrhosis of the liver; mesangial cell-proliferative diseases; injuries of the nerve tissue; and for inhibiting the re-occlusion of vessels after balloon catheter treatment, for use in vascular prosthetics or after inserting mechanical devices for holding vessels open, such as, e.g., stents, as immunosuppressants, as an aid in scar-free wound healing, and for treating age spots and contact dermatitis.

Preferably, the invention relates to the use of compounds of the formula I, or pharmaceutically acceptable salts thereof, in the treatment of solid tumors as mentioned herein.

### Process of Manufacture

A compound of formula I can be prepared analogously to methods that, for other compounds, are in principle known in the art, so that for the novel compounds of the formula I the process is novel as analogy process, preferably by reacting an isocyanate compound of the formula II,

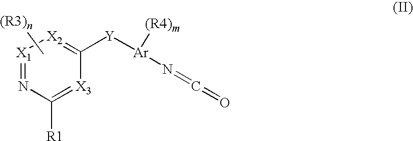

wherein R1, R3, R4, X₁, X₂, X₃, Ar, n and m are as defined for a compound of the formula I, with an amino compound of the formula III,

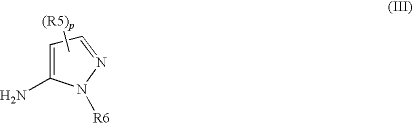

wherein R5, R6 and p are as defined for a compound of the formula I, and, if desired, transforming an obtainable compound of formula I into a different compound of formula I, transforming a salt of an obtainable compound of formula I into the free compound or a different salt, transforming an obtainable free compound of formula I into a salt thereof, and/or separating an obtainable mixture of isomers of a compound of formula I into individual isomers;
where in any starting materials functional groups that shall not take part in the reaction may be present in protected form and protecting groups are removed to obtain a compound of the formula I.

Preferably, the reaction between a compound of the formula II and a compound of the formula III takes place in an appropriate solvent, e.g. an ether, such as a cyclic ether, e.g. tetrahydrofurane, or an aliphatic ether, e.g. diethylether, or a mixture thereof, preferably at temperatures from −10 to 60° C., e.g. from 15 to 45° C.

### Optional Reactions and Conversions

Compounds of the formula I, or protected forms thereof directly obtained according to any one of the preceding procedures or after introducing protecting groups anew, which are included subsequently as starting materials for conversions as well even if not mentioned specifically, can be converted into different compounds of the formula I according to known procedures, where required followed removal of protecting groups.

For example, halogen or other potential leaving groups, such as C₁-C₇-alkylsulfonyl, (C₁-C₇-alkyl or unsubstituted)-phenylsulfonyl, can be converted into an azido group which can then be reduced into amino; thus, e.g. for the synthesis of a compound of the formula I wherein R1 is amino and R2, R3, R4, R5, R6, X₁, X₂, X₃, Y, Ar, m, n and o are as defined for a compound of the formula I, a corresponding halo or other leaving group at the position of R1 in a compound of the formula I can be converted by reaction with a (preferably alkali) metal azide, e.g. sodium azide, in an appropriate solvent, e.g. an N,N-di-C₁-C₇-alkyl-C₁-C₇-alkanoylamide, e.g. N,N-dimethylformamide, at customary temperatures, e.g. from 0° C. to the reflux temperature of the reaction mixture, e.g. from 20 to 75° C. to give a compound of the formula IV,

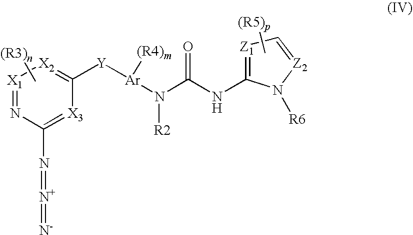

wherein R2, R3, R4, R5, R6, X₁, X₂, X₃, Y. Ar, m, n and o are as defined for a compound of the formula I, which can then be reduced to give a corresponding compound of the formula I with R1=NH₂. The reduction preferably takes place by hydrogenation, e.g. with hydrogen in the presence of a noble metal catalyst, e.g. palladium, rhodium or the like which may preferably be present on a carrier material, such as charcoal, in an appropriate solvent, e.g. methanol or ethanol, and at customary temperatures, e.g. from −10 to 60° C., e.g. et room temperature.

In a compound of the formula I where halogen or other potential leaving groups, such as C₁-C₇-alkylsulfonyl, (C₁-C₇-alkyl or unsubstituted)-phenylsulfonyl, are present, these can be converted into substituted amino by reaction with a corresponding amine; thus, for the synthesis of a compound of the formula I wherein R1 is substituted amino and R2, R3, R4, R5, R6, X₁, X₂, X₃, Y, Ar, m, n and o are as defined for a compound of the formula I, this conversion can be achieved by reacting a compound of the formula V,

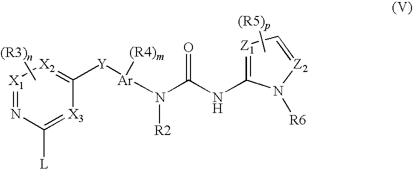

wherein L is a leaving group and R2, R3, R4, R5, R6, X₁, X₂, X₃, Y, Ar, m, n and o are as defined for a compound of the formula I, with an amino compound of the formula VI,

Ra—NH—Rb (VI)
wherein each of Ra and Rb is an amino substituent as defined for substituted amino in a compound of the formula I, with the proviso that up to one of Ra and Rb may be hydrogen, to give a corresponding compound of the formula I. This reaction preferably takes place under customary conditions, e.g. in an appropriate solvent, such as an alcohol, e.g. ethanol, at customary temperatures, e.g. from −10 to 50° C.

In the examples, appropriate reaction conditions can be found that may be used for analogous conversions of different compounds of the formula I.

Salts of compounds of formula I having at least one salt-forming group may be prepared in a manner known per se. For example, salts of compounds of formula I having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of formula I are obtained in customary manner, e.g. by treating the compounds with an acid or a suitable anion exchange reagent. Internal salts of compounds of formula I containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

A salt of a compound of the formula I can be converted in customary manner into the free compound; metal and ammonium salts can be converted, for example, by treatment with suitable acids, and acid addition salts, for example, by treatment with a suitable basic agent. In both cases, suitable ion exchangers may be used.

Stereoisomeric mixtures, e.g. mixtures of diastereomers, can be separated into their corresponding isomers in a manner known per se by means of appropriate separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

Intermediates and final products can be worked up and/or purified according to standard methods, e.g. using chromatographic methods, distribution methods, (re-) crystallization, and the like.

### Starting Materials

Starting Materials, including intermediates, for compounds of the formula I, such as the compounds of the formulae II and III, can be prepared, for example, according to methods that are known in the art, according to methods described in the examples or methods analogous to those described in the examples, and/or they are known or commercially available.

In the subsequent description of starting materials and intermediates and their synthesis, R1, R2, R3, R4, R5, R6, X₁, X₂, X₃, Ar, Y, Z₁, Z₂, m, n and p have the meanings given above or in the Examples for the respective starting materials or intermediates, if not indicated otherwise directly or by the context. Protecting groups, if not specifically mentioned, can be introduced and removed at appropriate steps in order to prevent functional groups, the reaction of which is not desired in the corresponding reaction step or steps, employing protecting groups, methods for their introduction and their removal are as described above or below, e.g. in the references mentioned under “General Process Conditions”. The person skilled in the art will readily be able to decide whether and which protecting groups are useful or required.

For example, a compound of the formula II can be obtained by converting an amino compound of the formula VII,

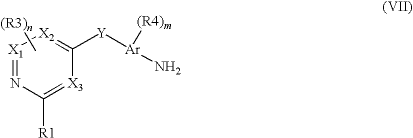

in the presence of e.g. phosgene in an appropriate solvent, such as a hydrocarbon, e.g. toluene, and/or an halogenated hydrocarbon, such as methylene chloride, at temperatures from e.g. −25° C. to the distillation temperature of the reaction mixture, e.g. up to about 110° C., to yield the corresponding isocyanate of the formula II.

A compound of the formula VII can, for example, be obtained from a corresponding compound bearing a nitro group instead of the amino group in formula VII by reduction, e.g. by hydrogenation in the presence of an appropriate transition metal catalyst, such as Raney-Ni or Raney-Co, in an appropriate solvent, e.g. an alcohol, such as methanol, and/or an ether, such as tetrahydrofurane, e.g. at temperatures from 0 to 50° C., for example at room temperature.

Such a nitro compound wherein Y is oxy, imino or thio can, for example, be obtained by reaction of a compound of the formula VIII,

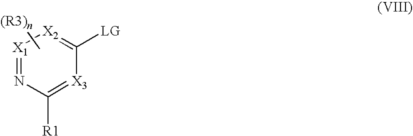

wherein LG is a leaving group, e.g. halo, such as chloro or bromo, with a compound of the formula IX;

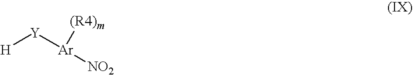

wherein Y is oxy, thio or imino, in the absence or presence of a base, such as an alkali metal hydroxide, e.g. sodium hydroxide, in the presence of an appropriate solvent, e.g. water and/or a ketone, such as acetone, at temperatures from e.g. 30 to 70° C.

A compound of the formula III wherein one R5 (p=1) is present that is unsubstituted or substituted alkyl and is bound in 5-position of the pyrazole ring in formula III can, for example, be prepared by reacting a nitrile compound of the formula X,

R5-C(═O)—CH₂—CN (X)

wherein R5 is as just defined, with a hydrazine compound of the formula XI,

R6-NH—NH₂ (XI)

wherein R6 is as defined for a compound of the formula I, e.g. in an appropriate solvent, such as a hydrocarbon, e.g. toluene, at temperatures e.g. in the range from 0° C. to the reflux temperature of the reaction mixture.

A compound of the formula III wherein R6 is heterocyclyl or aryl with a carboxyl (—COOH) group can be converted into a corresponding starting material of the formula III wherein instead of the carboxyl group an N-mono- or N,N-disubstituted carbamoyl group is present by reaction first with the corresponding N-mono-N,N-disubstituted amine (with or without addition of a tertiary nitrogen base) in the presence of an appropriate solvent, e.g. an ether, for example tetrahydrofurane, in the presence of a coupling agent, such as N-(3-dimethylaminopropyl)-N-ethylcarbodiimide dihydrochloride. For the synthesis of a starting material of the formula III wherein R6 is heterocyclyl or aryl with an N-mono- or N,N-disubstituted aminomethyl substituent, the carbonyl group in N-mono- or N,N-disubstituted carbamoyl just mentioned can be reduced to a methylene group, e.g. by reduction with borane in an appropriate solvent, such as an ether, e.g. tetrahydrofurane, for example at temperatures from 0 to 50° C., e.g. at room temperature.

Other starting materials are known in the art, commercially available and/or can be prepared according to standard procedures, e.g. in analogy to or by methods described in the Examples.

### General Process Conditions

The following applies in general to all processes mentioned hereinbefore and hereinafter, while reaction conditions specifically mentioned above or below are preferred:

In any of the reactions mentioned hereinbefore and hereinafter, protecting groups may be used where appropriate or desired, even if this is not mentioned specifically, to protect functional groups that are not intended to take part in a given reaction, and they can be introduced and/or removed at appropriate or desired stages. Reactions comprising the use of protecting groups are therefore included as possible wherever reactions without specific mentioning of protection and/or deprotection are described in this specification.

Within the scope of this disclosure only a readily removable group that is not a constituent of the particular desired end product of formula I is designated a “protecting group”, unless the context indicates otherwise. The protection of functional groups by such protecting groups, the protecting groups themselves, and the reactions appropriate for their removal are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie” (Methods of Organic Chemistry), Houben Weyl, 4th edition, Volume 15/1, Georg Thieme Verlag, Stuttgart 1974, in H.-D. Jakubke and H. Jeschkeit, “Aminosäuren, Peptide, Proteine” (Amino acids, Peptides, Proteins), Verlag Chemie, Weinheim, Deerfield Beach, and Basel 1982, and in Jochen Lehmann, “Chemie der Kohlenhydrate: Monosaccharide und Derivate” (Chemistry of Carbohydrates: Monosaccharides and Derivatives), Georg Thieme Verlag, Stuttgart 1974. A characteristic of protecting groups is that they can be removed readily (i.e. without the occurrence of undesired secondary reactions) for example by solvolysis, reduction, photolysis or alternatively under physiological conditions (e.g. by enzymatic cleavage).

All the above-mentioned process steps can be carried out under reaction conditions that are known per se, preferably those mentioned specifically, in the absence or, customarily, in the presence of solvents or diluents, preferably solvents or diluents that are inert towards the reagents used and dissolve them, In the absence or presence of catalysts, condensation or neutralizing agents, for example ion exchangers, such as cation exchangers, e.g. in the H⁺ form, depending on the nature of the reaction and/or of the reactants at reduced, normal or elevated temperature, for example in a temperature range of from about −100° C. to about 190° C., preferably from approximately −80° C. to approximately 150° C. for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofurane or dioxane, liquid aromatic hydrocarbons, such as benzene or toluene, alcohols, such as methanol, ethanol or 1- or 2-propanol, nitriles, such as acetonitrile, halogenated hydrocarbons, e.g. as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of these, for example aqueous solutions, unless otherwise indicated in the description of the processes. Such solvent mixtures may also be used in working up, for example by chromatography or partitioning.

Intermediates and final products can be worked up and/or purified according to standard methods, e.g. using chromatographic methods, distribution methods, (re-) crystallization, distillation (under normal or reduced pressure), steam distillation and the like.

The invention relates also to those forms of the process in which a compound obtainable as intermediate at any stage of the process is used as starting material and the remaining process steps are carried out, or in which a starting material is formed under the reaction conditions or is used in the form of a derivative, for example in protected form or in the form of a salt, or a compound obtainable by the process according to the invention is produced under the process conditions and processed further in situ. In the process of the present invention those starting materials are preferably used which result in compounds of formula I described as being preferred. Special preference is given to reaction conditions that are identical or analogous to those mentioned in the Examples.

### PREFERRED EMBODIMENTS ACCORDING TO THE INVENTION

In the following preferred embodiments as well as in preceding and following embodiments of more general scope, any one or more or all general expressions can be replaced by the corresponding more specific definitions provided above and below, thus yielding stronger preferred embodiments of the invention.

The invention relates preferably to a compound of the formula I, wherein

R1 is selected from the group consisting of amino, N-mono- or N,N-di-C₁-C₇-alkylamino, C₁-C₇-alkanoylamino, C₁-C₇-alkoxy-C₁-C₇-alkanoylamino, C₁-C₇-alkoxycarbonylamino, N-mono- or N,N-di-C₁-C₇-alkoxy-C₁-C₇alkylamino, N-mono- or N,N-di-(C₁-C₇-alkyl)-amino-C₁-C₇-alkylamino, optionally C₁-C₇-alkyl-substituted pyrrolidinyl-C₁-C₇-alkylamino, optionally C₁-C₇-alkyl-substituted morpholinyl-C₁-C₇-alkylamino, optionally C₁-C₇-alkyl-substituted piperazinyl-C₁-C₇-alkylamino and optionally C₁-C₇-alkyl-substituted piperidinyl-C₁-C₇-alkylamino;
R2 is C₁-C₇-alkyl or hydrogen;
n is 0 or 1;
m is 0 or 1;
p is 0, 1 or, 2;
each of R3 and R4, independently of the other, is hydrogen, C₁-C₇-alkyl, halo, hydroxy, C₁-C₇-alkoxy, nitro, amino, N-mono- or N,N-di-C₁-C₇-alkylamino, carboxy, C₁-C₇-alkoxycarbonyl, carbamoyl, N-mono- or N,N-di-C₁-C₇-alkyl-carbamoyl, sulfo, sulfamoyl or cyano;
R5 is C₁-C₇-alkyl, phenyl, furyl or C₃-C₈-cycloalkyl;
R6 is phenyl which is unsubstituted or substituted by up to three substituents independently selected from the group consisting of C₁-C₇-alkyl, halo-C₁-C₇-alkyl, C₁-C₇-alkoxy, C₁-C₇-alkylsulfonyl, C₁-C₇-alkoxy-C₁-C₇-alkoxy-C₁-C₇-alkoxy, C₁-C₇-alkoxycarbonyl, benzyloxy, cyano, N-mono- or N,N-di-(C₁-C₇-alkyl)-amino-C₁-C₇-alkyl, N—(N′-mono- or N′,N′-di-(C₁-C₇-alkyl)-amino-C₁-C₇-alkyl)-N—(C₁-C₇-alkyl)-amino-C₁-C₇-alkyl, pyrrolidino-C₁-C₇-alkyl, piperidino-C₁-C₇-alkyl, morpholino-C₁-C₇-alkyl, N—C₁-C₇-alkyl-piperazino-C₁-C₇-alkyl, N-mono- or N,N-di-(C₁-C₇-alkyl)-amino-substituted pyrrolidino-C₁-C₇-alkyl, pyrrolidino-carbonyl, piperidino-carbonyl, morpholinocarbonyl, N—C₁-C₇-alkyl-piperazino-carbonyl, N-mono- or N,N-di-(C₁-C₇-alkyl)-amino-substituted pyrrolidino-carbonyl and halo; X₁ and X₃ are CH and X₂ is N;
Y is imino (—NH—), thio (—S—) or oxy (—O—);
Ar is phenylene;
and one of Z₁ and Z₂ is N, the other is CH;
or a pharmaceutically acceptable salt thereof, or the USE of such compound of the formula I or a pharmaceutically acceptable salt thereof.

A more preferred embodiment of the invention relates to a compound of the formula I, wherein,

R1 is selected from the group consisting of amino, methylamino, (1-methyl-pyrrolidin-2-yl)-ethylamino, (pyrrolidin-1-yl)-ethylamino, (morpholin-4-yl)-ethylamino, (morpholin-4-yl)-propylamino, 2-(N,N-dimethyl-amino)-ethylamino, (4-methyl-piperazin-1-yl)-propylamino, (2-methyl-piperidin-1-yl)-propylamino, (1-methyl-piperidin-4-yl)-amino, (1-ethyl-pyrrolidin-2-yl)-methylamino, carbamic acid methylester and 2-methoxy acetamide;
R2 is hydrogen;
n is 0;
m is 0 or 1;
p is 1;
R4 is hydrogen, methoxy or fluoro;
R5 is tert-butyl which is bound in position 5 of the pyrazolyl ring in formula I;
R6 is phenyl which is unsubstituted or substituted by a substituent selected from the group consisting of methyl, isopropyl, trifluoromethyl, methoxy, methyl-sulfonyl, methoxy-ethoxy-methoxy, methoxycarbonyl, benzyloxy, cyano, N,N-dimethylaminomethyl, N—(N′,N′-dimethylaminopropyl)-N-methyl-aminomethyl, morpholinomethyl, 4-methyl-piperazinomethyl, N,N-dimethyl-amino-pyrrolidino-methyl, morpholinocarbonyl, 4-methyl- or 4-isopropyl-piperazinocarbonyl, N,N-dimethyl-amino-pyrrolidino-carbonyl, fluoro, chloro and bromo;

### X₁ and X₃ are CH and X₂ is N;

### Y is oxy (—O—);

Ar is 1,4-phenylene;

### Z₁ is CH;

### Z₂ is N;

or a pharmaceutically acceptable salt thereof, or the USE of such compound of the formula I or a pharmaceutically acceptable salt thereof.

The invention furthermore relates preferably to a compound of the formula I, wherein

R1 is amino, C₁-C₇-alkanoylamino, mono- or di-[C₁-C₇-alkyl and/or C₁-C₇-alkoxy-C₁-C₇alkyl and/or (mono- or di-(C₁-C₇-alkyl)-amino-C₁-C₇-alkyl]-amino or mono- or di-(naphthyl- or phenyl-C₁-C₇-alkyl)-amino, or (where the binding nitrogen forms part of a ring) piperidino, morpholino, thiomorpholino, N—C₁-C₇-alkyl-piperazino, or N-mono- or N,N-di-(C₁-C₇-alkyl-substituted or unsubstituted pyrrolidino or substituted amino, especially amino or N-mono- or N,N-di-C₁-C₇-alkylamino;
R2 is C₁-C₇-alkyl or preferably hydrogen;
n is 0 or 1;
m is 0 or 1;
p is 0, 1 or, 2;
each of R3 and R4, independently of the other, is C₁-C₇-alkyl, halo, hydroxy, C₁-C₇-alkoxy, nitro, amino, N-mono- or N,N-di-lower alkylamino, carboxy, C₁-C₇-alkoxycarbonyl, carbamoyl, N-mono- or N,N-di-C₁-C₇-alkyl-carbamoyl, sulfo, sulfamoyl or cyano;
R5 is selected from the groups mentioned for R3 and R4 or is phenyl, naphthyl or C₃-C₈-cycloalkyl;
R6 is phenyl or naphthyl which are unsubstituted or substituted by up to three substituents independently selected from the group consisting of C₁-C₇-alkyl, such as methyl or isopropyl, halo-C₁-C₇-alkyl, such as trifluoromethyl, N-mono- or N,N-di-(C₁-C₇-alkyl and/or mono-C₁-C₇-alkoxy-C₁-C₇alkyl and/or (mono- or di-(C₁-C₇-alkyl)amino-C₁-C₇-alkyl)-amino-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, N-mono- or N,N-di-(C₁-C₇-alkyl)-amino-substituted or unsubstituted pyrrolidino-C₁-C₇-alkyl, pyrrolidino-carbonyl, piperidino-carbonyl, morpholinocarbonyl, thiomorpholinocarbonyl, N—C₁-C₇-alkyl-piperazino-carbonyl, N-mono- or N,N-di-(C₁-C₇-alkyl)-amino-substituted or unsubstituted pyrrolidino-carbonyl, halo, such as fluoro, chloro or bromo, N-mono- or N,N-di-(C₁-C₇-alkyl and/or mono-C₁-C₇-alkoxy-C₁-C₇-alkyl and/or (mono- or di-(C₁-C₇-alkyl)-amino-C₁-C₇-alkyl)-aminocarbonyl,

### X₁ and X₃ are CH and X₂ is CH or N;

Y is imino (—NH—), thio (—S—) or preferably oxy (—O—);
Ar is phenylene, especially 1,4-phenylene;
and one of Z₁ and Z₂ is N, the other is CH;
or a pharmaceutically acceptable salt thereof, or the USE of such compound of the formula I or a pharmaceutically acceptable salt thereof.

The invention furthermore relates preferably to a compound of the formula I, wherein

R1 is amino or methylamino;
R2 is hydrogen;
n is 0;
m is 0 or 1;
p is 1;
R4 is methoxy;
R5 is branched C₁-C₇-alkyl or is C₃-C₈-cycloalkyl and is bound preferably in position 5 of the pyrazolyl ring in formula I;
R6 is phenyl is unsubstituted or substituted by a substituent selected from the group consisting of methyl, isopropyl, trifluoromethyl, N,N-dimethylaminomethyl, N—(N′,N′-dimethylaminopropyl)-N-methyl-aminomethyl, morpholinomethyl, 4-methyl-piperazinomethyl, N,N-dimethyl-amino-pyrrolidino-methyl, morpholinomethyl, 4-methyl- or 4-isopropyl-piperazinomethyl, N,N-dimethyl-amino-pyrrolidino-methyl and halo, such as fluoro, chloro or bromo;

### X₁ and X₃ are CH and X₂ is N;

### Y is oxy (—O—); a

Ar is 1,4-phenylene;
and one of Z₁ and Z₂ is N, the other is CH;
or a pharmaceutically acceptable salt thereof, or the USE of such compound of the formula I or a pharmaceutically acceptable salt thereof.

Most preferred is a compound of the formula I, or a (preferably pharmaceutically acceptable) salt thereof, as exemplified herein-below under ‘Examples’, or its USE as defined above.

### Pharmaceutical Compositions

The invention relates also to pharmaceutical compositions comprising a (preferably novel) compound of formula I, to their use in the therapeutic (in a broader aspect of the invention also prophylactic) treatment or a method of treatment of a disease or disorder that depends on inadequate protein (especially Tie-2) kinase activity, especially the preferred disorders or diseases mentioned above, to the compounds for said use and to pharmaceutical preparations and their manufacture, especially for said uses. More generally, pharmaceutical preparations are useful in case of compounds of the formula I.

The pharmacologically acceptable compounds of the present invention may be present in or employed, for example, for the preparation of pharmaceutical compositions that comprise an effective amount of a compound of the formula I, or a pharmaceutically acceptable salt thereof, as active ingredient together or in admixture with one or more inorganic or organic, solid or liquid, pharmaceutically acceptable carriers (carrier materials).

The invention relates also to a pharmaceutical composition that is suitable for administration to a warm-blooded animal, especially a human (or to cells or cell lines derived from a warm-blooded animal, especially a human, e.g. lymphocytes), for the treatment (this, in a broader aspect of the invention, also including prevention of (=prophylaxis against)) a disease that responds to inhibition of protein (especially Tie-2) kinase activity, comprising an amount of a compound of formula I or a pharmaceutically acceptable salt thereof, preferably which is effective for said inhibition, together with at least one pharmaceutically acceptable carrier.

The pharmaceutical compositions according to the invention are those for enteral, such as nasal, rectal or oral, or parenteral, such as intramuscular or intravenous, administration to warm-blooded animals (especially a human), that comprise an effective dose of the pharmacologically active ingredient, alone or together with a significant amount of a pharmaceutically acceptable carrier. The dose of the active ingredient depends on the species of warm-blooded animal, the body weight, the age and the individual condition, individual pharmacokinetic data, the disease to be treated and the mode of administration.

The invention relates also to method of treatment for a disease that responds to inhibition of a disease that depends on inadequate activity of a protein (especially Tie-2) kinase; which comprises administering a prophylactically or especially therapeutically effective amount of a compound of formula I, or a pharmaceutically acceptable salt thereof, especially to a warm-blooded animal, for example a human, that, on account of one of the mentioned diseases, requires such treatment.

The dose of a compound of the formula I or a pharmaceutically acceptable salt thereof to be administered to warm-blooded animals, for example humans of approximately 70 kg body weight, preferably is from approximately 3 mg to approximately 10 g, more preferably from approximately 10 mg to approximately 1.5 g, most preferably from about 100 mg to about 1000 mg/person/day, divided preferably into 1-3 single doses which may, for example, be of the same size. Usually, children receive half of the adult dose.

The pharmaceutical compositions comprise from approximately 1% to approximately 95%, preferably from approximately 20% to approximately 90%, active ingredient. Pharmaceutical compositions according to the invention may be, for example, in unit dosage form, such as in the form of ampoules, vials, suppositories, dragées, tablets or capsules.

The pharmaceutical compositions of the present invention are prepared in a manner known per se, for example by means of conventional dissolving, lyophilizing, mixing, granulating or confectioning processes.

Solutions of the active ingredient, and also suspensions, and especially isotonic aqueous solutions or suspensions, are preferably used, it being possible, for example in the case of lyophilized compositions that comprise the active ingredient alone or together with a carrier, for example mannitol, for such solutions or suspensions to be produced prior to use. The pharmaceutical compositions may be sterilized and/or may comprise excipients, for example preservatives, stabilizers, wetting and/or emulsifying agents, solubilizers, salts for regulating the osmotic pressure and/or buffers, and are prepared in a manner known per se, for example by means of conventional dissolving or lyophilizing processes. The said solutions or suspensions may comprise viscosity-increasing substances, such as sodium carboxymethylcellulose, carboxymethylcellulose, dextran, polyvinylpyrrolidone or gelatin.

Suspensions in oil comprise as the oil component the vegetable, synthetic or semi-synthetic oils customary for injection purposes. There may be mentioned as such especially liquid fatty acid esters that contain as the acid component a long-chained fatty acid having from 8-22, especially from 12-22, carbon atoms, for example lauric acid, tridecylic acid, myristic acid, pentadecylic acid, palmitic acid, margaric acid, stearic acid, arachidic acid, behenic acid or corresponding unsaturated acids, for example oleic acid, elaidic acid, erucic acid, brasidic acid or linoleic acid, if desired with the addition of antioxidants, for example vitamin E, β-carotene or 3,5-di-tert-butyl-4-hydroxytoluene. The alcohol component of those fatty acid esters has a maximum of 6 carbon atoms and is a mono- or poly-hydroxy, for example a mono-, di- or tri-hydroxy, alcohol, for example methanol, ethanol, propanol, butanol or pentanol or the isomers thereof, but especially glycol and glycerol. The following examples of fatty acid esters are therefore to be mentioned: ethyl oleate, isopropyl myristate, isopropyl palmitate, “Labrafil M 2375” (polyoxyethylene glycerol trioleate, Gattefossé, Paris), “Miglyol 812” (triglyceride of saturated fatty acids with a chain length of C8 to C12, Hüls AG, Germany), but especially vegetable oils, such as cottonseed oil, almond oil, olive oil, castor oil, sesame oil, soybean oil and groundnut oil.

The injection or infusion compositions are prepared in customary manner under sterile conditions; the same applies also to introducing the compositions into ampoules or vials and sealing the containers.

Pharmaceutical compositions for oral administration can be obtained by combining the active ingredient with solid carriers, if desired granulating a resulting mixture, and processing the mixture, if desired or necessary, after the addition of appropriate excipients, into tablets, dragée cores or capsules. It is also possible for them to be incorporated into plastics carriers that allow the active ingredients to diffuse or be released in measured amounts.

Suitable carriers are especially fillers, such as sugars, for example lactose, saccharose, mannitol or sorbitol, cellulose preparations and/or calcium phosphates, for example tri-calcium phosphate or calcium hydrogen phosphate, and binders, such as starch pastes using for example corn, wheat, rice or potato starch, gelatin, tragacanth, methylcellulose, hydroxypropylmethylcellulose, sodium carboxymethylcellulose and/or polyvinylpyrrolidone, and/or, if desired, disintegrators, such as the above-mentioned starches, and/or carboxymethyl starch, crosslinked polyvinylpyrrolidone, agar, alginic acid or a salt thereof, such as sodium alginate. Excipients are especially flow conditioners and lubricants, for example silicic acid, talc, stearic acid or salts thereof, such as magnesium or calcium stearate, and/or polyethylene glycol. Dragée cores are provided with suitable, optionally enteric, coatings, there being used, inter alia, concentrated sugar solutions which may comprise gum arabic, talc, polyvinylpyrrolidone, polyethylene glycol and/or titanium dioxide, or coating solutions in suitable organic solvents, or, for the preparation of enteric coatings, solutions of suitable cellulose preparations, such as ethylcellulose phthalate or hydroxypropylmethylcellulose phthalate. Capsules are dry-filled capsules made of gelatin and soft sealed capsules made of gelatin and a plasticizer, such as glycerol or sorbitol. The dry-filled capsules may comprise the active ingredient in the form of granules, for example with fillers, such as lactose, binders, such as starches, and/or glidants, such as talc or magnesium stearate, and if desired with stabilizers. In soft capsules the active ingredient is preferably dissolved or suspended in suitable oily excipients, such as fatty oils, paraffin oil or liquid polyethylene glycols, it being possible also for stabilizers and/or antibacterial agents to be added. Dyes or pigments may be added to the tablets or dragée coatings or the capsule casings, for example for identification purposes or to indicate different doses of active ingredient.

A compound of the formula I may also be used to advantage in combination with other anti-proliferative agents. Such antiproliferative agents include, but are not limited to aromatase inhibitors; antiestrogens; topoisomerase I inhibitors; topoisomerase II inhibitors; microtubule active agents; alkylating agents; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; bisphosphonates; biological response modifiers; antiproliferative antibodies; heparanase inhibitors; inhibitors of Ras oncogenic isoforms; telomerase inhibitors; proteasome inhibitors; agents used in the treatment of hematologic malignancies; compounds which target, decrease or inhibit the activity of Flt-3; Hsp90 inhibitors; and temozolomide (TEMODAL®).

The term “aromatase inhibitor” as used herein relates to a compound which inhibits the estrogen production, i.e. the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane can be administered, e.g., in the form as it is marketed, e.g. under the trademark AROMASIN. Formestane can be administered, e.g., in the form as it is marketed, e.g. under the trademark LENTARON. Fadrozole can be administered, e.g., in the form as it is marketed, e.g. under the trademark AFEMA. Anastrozole can be administered, e.g., in the form as it is marketed, e.g. under the trademark ARIMIDEX. Letrozole can be administered, e.g., in the form as it is marketed, e.g. under the trademark FEMARA or FEMAR. Aminoglutethimide can be administered, e.g., in the form as it is marketed, e.g. under the trademark ORIMETEN. A combination of the invention comprising a chemotherapeutic agent which is an aromatase inhibitor is particularly useful for the treatment of hormone receptor positive tumors, e.g. breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen can be administered, e.g., in the form as it is marketed, e.g. under the trademark NOLVADEX. Raloxifene hydrochloride can be administered, e.g., in the form as it is marketed, e.g. under the trademark EVISTA. Fulvestrant can be formulated as disclosed in U.S. Pat. No. 4,659,516 or it can be administered, e.g., in the form as it is marketed, e.g. under the trademark FASLODEX. A combination of the invention comprising a chemotherapeutic agent which is an antiestrogen is particularly useful for the treatment of estrogen receptor positive tumors, e.g. breast tumors.

The term “anti-androgen” as used herein relates to any substance which is capable of inhibiting the biological effects of androgenic hormones and includes, but is not limited to, bicalutamide (CASODEX), which can be formulated, e.g. as disclosed in U.S. Pat. No. 4,636,505.

The term “gonadorelin agonist” as used herein includes, but is not limited to abarelix, goserelin and goserelin acetate. Goserelin is disclosed in U.S. Pat. No. 4,100,274 and can be administered, e.g., in the form as it is marketed, e.g. under the trademark ZOLADEX. Abarelix can be formulated, e.g. as disclosed in U.S. Pat. No. 5,843,901.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148 (compound A1 in WO99/17804). Irinotecan can be administered, e.g. in the form as it is marketed, e.g. under the trademark CAMPTOSAR. Topotecan can be administered, e.g., in the form as it is marketed, e.g. under the trademark HYCAMTIN.

The term “topoisomerase II inhibitor” as used herein includes, but is not limited to the anthracyclines such as doxorubicin (including liposomal formulation, e.g. CAELYX), daunorubicin, epirubicin, idarubicin and nemorubicin, the anthraquinones mitoxantrone and losoxantrone, and the podophillotoxines etoposide and teniposide. Etoposide can be administered, e.g. in the form as it is marketed, e.g. under the trademark ETOPOPHOS. Teniposide can be administered, e.g. in the form as it is marketed, e.g. under the trademark VM 26-BRISTOL. Doxorubicin can be administered, e.g. in the form as it is marketed, e.g. under the trademark ADRIBLASTIN or ADRIAMYCIN. Epirubicin can be administered, e.g. in the form as it is marketed, e.g. under the trademark FARMORUBICIN. Idarubicin can be administered, e.g. in the form as it is marketed, e.g. under the trademark ZAVEDOS. Mitoxantrone can be administered, e.g. in the form as it is marketed, e.g. under the trademark NOVANTRON.

The term “microtubule active agent” relates to microtubule stabilizing, microtubule destabilizing agents and microtublin polymerization inhibitors including, but not limited to taxanes, e.g. paclitaxel and docetaxel, vinca alkaloids, e.g., vinblastine, especially vinblastine sulfate, vincristine especially vincristine sulfate, and vinorelbine, discodermolides, cochicine and epothilones and derivatives thereof, e.g. epothilone B or a derivative thereof. Paclitaxel may be administered e.g. in the form as it is marketed, e.g. TAXOL. Docetaxel can be administered, e.g., in the form as it is marketed, e.g. under the trademark TAXOTERE. Vinblastine sulfate can be administered, e.g., in the form as it is marketed, e.g. under the trademark VINBLASTIN R.P. Vincristine sulfate can be administered, e.g., in the form as it is marketed, e.g. under the trademark FARMISTIN. Discodermolide can be obtained, e.g., as disclosed in U.S. Pat. No. 5,010,099. Also included are Epothilone derivatives which are disclosed in WO 98/10121, U.S. Pat. No. 6,194,181, WO 98/25929, WO 98/08849, WO 99/43653, WO 98/22461 and WO 00/31247. Especially preferred are Epothilone A and/or B.

The term “alkylating agent” as used herein includes, but is not limited to, cyclophosphamide, ifosfamide, melphalan or nitrosourea (BCNU or Gliadel). Cyclophosphamide can be administered, e.g., in the form as it is marketed, e.g. under the trademark CYCLOSTIN. Ifosfamide can be administered, e.g., in the form as it is marketed, e.g. under the trademark HOLOXAN.

The term “histone deacetylase inhibitors” or “HDAC inhibitors” relates to compounds which inhibit the histone deacetylase and which possess antiproliferative activity. This includes compounds disclosed in WO 02/22577, especially N-hydroxy-3-[4-[[(2-hydroxyethyl)[2-(1H-indol-3-yl)ethyl]-amino]methyl]phenyl]-2E-2-propenamide, N-hydroxy-3-[4-[[[2-(2-methyl-1H-indol-3-yl)-ethyl]-amino]methyl]phenyl]-2E-2-propenamide and pharmaceutically acceptable salts thereof. It further especially includes Suberoylanilide hydroxamic acid (SAHA).

The term “antineoplastic antimetabolite” includes, but is not limited to, 5-Fluorouracil or 5-FU, capecitabine, gemcitabine, DNA demethylating agents, such as 5-azacytidine and decitabine, methotrexate and edatrexate. Capecitabine can be administered, e.g., in the form as it is marketed, e.g. under the trademark XELODA Gemcitabine can be administered, e.g., in the form as it is marketed, e.g. under the trademark GEMZAR. Also included is the monoclonal antibody trastuzumab which can be administered, e.g., in the form as it is marketed, e.g. under the trademark HERCEPTIN.

The term “platin compound” as used herein includes, but is not limited to, carboplatin, cis-platin, cisplatinum and oxaliplatin. Carboplatin can be administered, e.g., in the form as it is marketed, e.g. under the trademark CARBOPLAT. Oxaliplatin can be administered, e.g., in the form as it is marketed, e.g. under the trademark ELOXATIN.

The term “compounds targeting/decreasing a protein or lipid kinase activity and further anti-angiogenic compounds” as used herein includes, but is not limited to: protein tyrosine kinase and/or serine and/or threonine kinase inhibitors or lipid kinase inhibitors, e.g.:

a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, e.g. a N-phenyl-2-pyrimidine-amine derivative, e.g. imatinib, SU101, SU6668, and GFB-111;
b) compounds targeting, decreasing or inhibiting the activity of the fibroblast growth factor-receptors (FGFR);
c) compounds targeting, decreasing or inhibiting the activity of the insulin-like growth factor receptor 1 (IGF-1R), such as compounds which target, decrease or inhibit the activity of IGF-IR, especially compounds which inhibit the IGF-1R receptor, such as those compounds disclosed in WO 02/092599;
d) compounds targeting, decreasing or inhibiting the activity of the Trk receptor tyrosine kinase family;
e) compounds targeting, decreasing or inhibiting the activity of the Axl receptor tyrosine kinase family;
f) compounds targeting, decreasing or inhibiting the activity of the c-Met receptor;
g) compounds targeting, decreasing or inhibiting the activity of the c-Kit receptor tyrosine kinases—(part of the PDGFR family), such as compounds which target, decrease or inhibit the activity of the c-Kit receptor tyrosine kinase family, especially compounds which inhibit the c-Kit receptor, e.g. imatinib;
h) compounds targeting, decreasing or inhibiting the activity of members of the c-Abl family and their gene-fusion products (e.g. BCR-Abl kinase), such as compounds which target, decrease or inhibit the activity of c-Abl family members and their gene fusion products, e.g. a N-phenyl-2-pyrimidine-amine derivative, e.g. imatinib; PD180970; AG957; NSC 680410; or PD173955 from ParkeDavis;
i) compounds targeting, decreasing or inhibiting the activity of members of the protein kinase C (PKC) and Raf family of serine/threonine kinases, members of the MEK, SRC, JAK, FAK, PDK and Ras/MAPK family members, or PI(3) kinase family, or of the PI(3)-kinase-related kinase family, and/or members of the cyclin-dependent kinase family (CDK) and are especially those staurosporine derivatives disclosed in U.S. Pat. No. 5,093,330, e.g. midostaurin; examples of further compounds include e.g. UCN-01, safingol, BAY 43-9006, Bryostatin 1, Perifosine; Ilmofosine; RO 318220 and RO 320432; GO 6976; Isis 3521; LY333531/LY379196; isochinoline compounds such as those disclosed in WO 00/09495; FTIs; PD184352 or QAN697 (a P13K inhibitor);
j) compounds targeting, decreasing or inhibiting the activity of a protein-tyrosine kinase, such as imatinib mesylate (GLIVEC/GLEEVEC) or tyrphostin. A tyrphostin is preferably a low molecular weight (Mr<1500) compound, or a pharmaceutically acceptable salt thereof, especially a compound selected from the benzylidenemalonitrile class or the S-arylbenzenemalonirile or bisubstrate quinoline class of compounds, more especially any compound selected from the group consisting of Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino)-benzoic acid adamantyl ester; NSC 680410, adaphostin);
k) compounds targeting, decreasing or inhibiting the activity of the epidermal growth factor family of receptor tyrosine kinases (EGFR, ErbB2, ErbB3, ErbB4 as homo- or heterodimers), such as compounds which target, decrease or inhibit the activity of the epidermal growth factor receptor family are especially compounds, proteins or antibodies which inhibit members of the EGF receptor tyrosine kinase family, e.g. EGF receptor, ErbB2, ErbB3 and ErbB4 or bind to EGF or EGF related ligands, and are in particular those compounds, proteins or monoclonal antibodies generically and specifically disclosed in WO 97/02266, e.g. the compound of ex. 39, or in EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/30347 (e.g. compound known as CP 358774), WO 96/33980 (e.g. compound ZD 1839) and WO 95/03283 (e.g. compound ZM105180); e.g. trastuzumab (HerpetinR), cetuximab, Iressa, OSI-774, CI-1033, EKB-569, GW-2016, E1.1, E2.4, E2.5, E6.2, E6.4, E2.11, E6.3 or E7.6.3, and 7H-pyrrolo-[2,3-d]pyrimidine derivatives which are disclosed in WO 03/013541; and
l) compounds targeting, decreasing or inhibiting the activity of the vascular endothelial growth factor-receptors (VEGFR), such as PTK-787 or Avastin.

Further anti-angiogenic compounds include compounds having another mechanism for their activity, e.g. unrelated to protein or lipid kinase inhibition e.g. thalidomide (THALOMID) and TNP-470 or RAD001.

Compounds which target, decrease or inhibit the activity of a protein or lipid phosphatase are e.g. inhibitors of phosphatase 1, phosphatase 2A, PTEN or CDC25, e.g. okadaic acid or a derivative thereof.

Compounds which induce cell differentiation processes are e.g. retinoic acid, α- γ- or δ-tocopherol or α-γ- or δ-tocotrienol.

The term “cyclooxygenase inhibitor” as used herein includes, but is not limited to, e.g. Cox-2 inhibitors, 5-alkyl substituted 2-arylaminophenylacetic acid and derivatives, such as celecoxib (CELEBREX), rofecoxib (VIOXX), etoricoxib, valdecoxib or a 5-alkyl-2-arylaminophenylacetic acid, e.g. 5-methyl-2-(2′-chloro-6′-fluoroanilino)phenyl acetic acid, lumiracoxib.

The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “bisphosphonates” as used herein includes, but is not limited to, etridonic, clodronic, tiludronic, pamidronic, alendronic, ibandronic, risedronic and zoledronic acid. “Etridonic acid” can be administered, e.g., in the form as it is marketed, e.g. under the trademark DIDRONEL. “Clodronic acid” can be administered, e.g., in the form as it is marketed, e.g. under the trademark BONEFOS. “Tiludronic acid” can be administered, e.g., in the form as it is marketed, e.g. under the trademark SKELID. “Pamidronic acid” can be administered, e.g. in the form as it is marketed, e.g. under the trademark AREDIA™. “Alendronic acid” can be administered, e.g., in the form as it is marketed, e.g. under the trademark FOSAMAX. “Ibandronic acid” can be administered, e.g., in the form as it is marketed, e.g. under the trademark BONDRANAT. “Risedronic acid” can be administered, e.g., in the form as it is marketed, e.g. under the trademark ACTONEL. “Zoledronic acid” can be administered, e.g. in the form as it is marketed, e.g. under the trademark ZOMETA.

The term “heparanase inhibitor” as used herein refers to compounds which target, decrease or inhibit heparin sulphate degradation. The term includes, but is not limited to, PI-88.

The term “biological response modifier” as used herein refers to a lymphokine or interferons, e.g. interferon γ.

The term “inhibitor of Ras oncogenic isoforms”, e.g. H-Ras, K-Ras, or N-Ras, as used herein refers to compounds which target, decrease or inhibit the oncogenic activity of Ras e.g. a “farnesyl transferase inhibitor”, e.g. L-744832, DK8G557 or R115777 (Zarnestra).

The term “telomerase inhibitor” as used herein refers to compounds which target, decrease or inhibit the activity of telomerase. Compounds which target, decrease or inhibit the activity of telomerase are especially compounds which inhibit the telomerase receptor, e.g. telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase are e.g. bengamide or a derivative thereof.

The term “proteasome inhibitor” as used herein refers to compounds which target, decrease or inhibit the activity of the proteasome. Compounds which target, decrease or inhibit the activity of the proteasome include e.g. PS-341 and MLN 341.

The term “matrix metalloproteinase inhibitor” or (“MMP inhibitor”) as used herein includes, but is not limited to collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “agents used in the treatment of hematologic malignancies” as used herein includes, but is not limited to FMS-like tyrosine kinase inhibitors e.g. compounds targeting, decreasing or inhibiting the activity of Flt-3; interferon, 1-b-D-arabinofuransylcytosine (ara-c) and bisulfan; and ALK inhibitors e.g. compounds which target, decrease or inhibit anaplastic lymphoma kinase.

The term “compounds which target, decrease or inhibit the activity of Flt-3” are especially compounds, proteins or antibodies which inhibit Flt-3, e.g. PKC412, midostaurin, a staurosporine derivative, SU11248 and MLN518.

The term “HSP90 inhibitors” as used herein includes, but is not limited to, compounds targeting, decreasing or inhibiting the Intrinsic ATPase activity of HSP90; degrading, targeting, decreasing or inhibiting the HSP90 client proteins via the ubiquitin proteasome pathway. Compounds targeting, decreasing or inhibiting the intrinsic ATPase activity of HSP90 are especially compounds, proteins or antibodies which inhibit the ATPase activity of HSP90 e.g., 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to trastuzumab (Herceptin™), Trastuzumab-DM1, erlotinib (Tarceva™), bevacizumab (Avastin™), rituximab (Rituxan®), PRO64553 (anti-CD40) and 2C4 Antibody. By antibodies is meant e.g. intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of formula I can be used in combination with standard leukemia therapies, especially in combination with therapies used for the treatment of AML. In particular, compounds of formula I can be administered in combination with e.g. farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

The structure of the active agents identified by code nos., generic or trade names may be taken from the actual edition of the standard compendium “The Merck Index” or from databases, e.g. Patents International (e.g. IMS World Publications).

The above-mentioned compounds, which can be used in combination with a compound of the formula I, can be prepared and administered as described in the art such as in the documents cited above.

A compound of the formula I may also be used to advantage in combination with known therapeutic processes, e.g., the administration of hormones or especially radiation.

A compound of formula I may in particular be used as a radiosensitizer, especially for the treatment of tumors which exhibit poor sensitivity to radiotherapy.

By “combination”, there is meant either a fixed combination in one dosage unit form, or a kit of parts for the combined administration where a compound of the formula I and a combination partner may be administered independently at the same time or separately within time intervals that especially allow that the combination partners show a cooperative, e.g. synergistic, effect, or by making use of administration schedules representing any combination thereof.

### EXAMPLES

The following Examples serve to illustrate the invention without limiting the scope thereof. Temperatures are measured in degrees Celsius. Unless otherwise indicated, the reactions take place at room temperature under N₂-atmosphere. The R_{f} values which indicate the ratio of the distance moved by each substance to the distance moved by the eluent front are determined on silica gel thin-layer plates 5×10 cm TLC plates, silica gel F₂₅₄ (Merck, Darmstadt, Germany) by thin-layer chromatography using the solvent systems indicated below.

### ABBREVIATIONS

Bn benzyl
brine saturated solution of NaCl in water
bs broad singlet
celite=Celite® (The Celite® Corporation)=filtering aid based on diatomaceous earth
conc. concentrated
d day(s)
DMAP N,N-dimethylaminopyridine
DMF N,N-dimethyl formamide
DMSO dimethylsulfoxide
ether diethylether
EDC N-(3-dimethylaminopropyl)-N-ethylcarbodiimide-hydrochloride
Et₃N triethylamine
EtOAc ethyl acetate
EtOH ethanol
Ex. Example
h hour(s)
HPLC high pressure liquid chromatography
l litre(s)
Me methyl
MEM methoxy-ethoxy-methyl
MeOH methanol
min minute(s)
m.p. melting point
MS mass spectrum
NEt₃ triethylamine
R_{f} ratio of fronts (TLC)
rt room temperature
Rₜ retention time (HPLC)
THF tetrahydrofuran (distilled from Na/benzophenone)
TFA trifluoroacetic acid
TLC thin layer chromatography
sat. saturated

### HPLC Conditions:

Rₜ^{A}: retention time [min] for System A: Linear gradient 20-100% CH₃CN (0.1% TFA) and H₂O (0.1% TFA) in 6 min+5 min 100% CH₃CN (0.1% TFA); detection at 215 nm, flow rate 1 ml/min at 25 or 35° C. Column: Nucleosil 120-3 C18 (70×4.0 mm).

Rₜ^{B}: retention time [min] for System B: Linear gradient 5-100% CH₃CN (0.1% TFA) and H₂O (0.1% TFA) in 4 min+0.6 min 100% CH₃CN (0.1% TFA); detection at 210 nm, flow rate 1.8 ml/min at 25 or 30° C. Column: XTerra MS 5 μM C18 (50×4.6 mm).

### Example 1

### 1-[4-(6-Amino-pyrimidin-4-yloxy)-phenyl-3-(5-tert-butyl-2-p-tolyl-2H-pyrazol-3-yl)-urea

A solution of 150 mg (0.31 mMol) of 1-[4-(6-azido-pyrimidin-4-yloxy)-phenyl]-3-(5-tert-butyl-2-p-tolyl-2H-pyrazol-3-yl)urea (Step 1.3) in 6 ml of MeOH is submitted to hydrogenation over Pd/C (10% Engelhardt 4505, 50 mg) at rt for 10 h. After completion of the reaction, the reaction mixture is filtered over a pad of Celite and washed with MeOH. The filtrates are combined and concentrated under reduced pressure. The crude product is suspended in cold CH₂Cl₂, filtered and dried under high vacuum to give the title compound as a white powder. MS: [M+1]⁺=458; ¹H-NMR (DMSO-d₆): 9.11 (s, HN), 8.37 (s, HN), 8.08 (s, 1H), 7.47 (d, J=9.0 Hz, 2H), 7.45 (d, J=8.61 Hz, 2H), 7.43 (d, J=8.61 Hz, 2H), 7.06 (d, J=9.0 Hz, 2H), 6.37 (s, 1H), 5.78 (s, 1H), 5.67 (s, 1H), 2.40 (s, 3H), 1.29 (s, 9H).

The starting material is prepared as follows:

### Step 1.1: 5-tert-Butyl-2-p-tolyl-2H-pyrazol-3-ylamine

The title compound is prepared according to a published literature procedure (see J. Med. Chem. 2002, 45, 2994-3008.) 3.5 g (27.8 mMol) of pivaloylacetonitrile are added to a solution of 3.4 g (27.8 mMol) p-tolylhydrazine in 50 mL of toluene at rt, and the resulting yellow solution is heated to and kept under reflux for 12 h. After completion, the reaction mixture is concentrated, and the resulting crude product is purified by flash chromatography (SiO₂, 100% CH₂Cl₂) to give the title compound as a yellow solid.

### Step 1.2: 1-(5-tert-Butyl-2-p-tolyl-2H-pyrazol-3-yl)-3-[4-(6-chloro-pyrimidin-4-yloxy)-phenyl]-urea

A solution of 200 mg (0.87 mMol) of 5-tert-butyl-2-p-tolyl-2H-pyrazol-3-ylamine in 9 mL of ether is treated with a solution of 211 mg (0.87 mMol) 4-chloro-6-(4-isocyanato-phenoxy)-pyrimidine (see step 1.6) in 3 mL THF at rt. The reaction is stirred for 2.5 h at rt and then warmed to and kept at 40° C. for 12 h. After completion, the reaction mixture is concentrated in vacuo, and the resulting crude product is purified by flash chromatography (SiO₂; MeOH/CH₂Cl₂; gradient 0-5% MeOH) to give the title compound as a white foam. MS: [M+1]⁺=478.

### Step 1.3: 1-[4-(6-Azido-pyrimidin-4yloxy)phenyl]-3-(5-tert-butyl-2-p-tolyl-2H-pyrazol-3yl)urea

A solution of 300 mg (0.63 mMol) of 1-(5-tert-butyl-2-p-tolyl-2H-pyrazol-3-yl)-3-[4-(6-chloro-pyrimidin-4-yloxy)-phenyl]urea in 5 mL DMF is treated with 82 mg (1.25 mMol) sodium azide at rt. The reaction is then heated to and kept at 70° C. for 1.5 h. After completion, the resulting reaction mixture is concentrated in vacuo. The residue is taken up in CH₂Cl₂ and washed twice with water. The organic layer is dried (Na₂SO₄) and concentrated, and the residual crude product is purified by flash chromatography (SiO₂, gradient MeOH/CH₂Cl₂ 1-5% MeOH) to give the title compound as a yellow solid. MS: [M+1]⁺=484; ¹H-NMR (DMSO-d₆): 9.13 (s, 1H), 8.38 (s, 1H), 7.48 (d, J=8.9 Hz, 2H), 7.43 (d, J=8.6 Hz, 2H), 7.36 (d, J=8.9 Hz, 2H), 7.13 (d, J=8.6 Hz, 2H), 6.38 (s, 1H), 2.40 (s, 3H), 1.30 (s, 9H).

### Step 1.4: 4-Chloro-6-(4-nitro-phenoxy)-pyrimidine

To an ice-cooled solution of 214 g (5.35 Mol) NaOH dissolved in 6.5 l of H₂O, 744 g (5.35 Mol) of 4-nitrophenol are added. Then a solution of 797 g (5.35 Mol) of 4,6-dichloro-pyrimidine in 6.5 l of acetone is added dropwise during 60 min, and the mixture is stirred for 18 h at 65° C. The reaction mixture is cooled to 10° C., the precipitated crude product filtered off and washed with 400 ml H₂O/acetone 1:1: m.p.: 127-128° C.

### Step 1.5: 4-(6-Chloro-pyrimidin-4-yl-oxy)-aniline

1095 g (4.3 Mol) of 4-chloro-6-(4-nitro-phenoxy)-pyrimidine dissolved in 10 l of MeOH/THF 2:1 are hydrogenated in the presence of 33 g Raney-Ni at rt for 4 h. The reaction solution is filtered and concentrated. Crystallization from EtOAc gives the title compound: ¹H-NMR (DMSO-d₆): 8.60 (s, 1H), 7.12 (s, 1H), 6.86 (d, 9 Hz, 2H), 6.57 (d, 9 Hz, 2H), 5.13 (s, 2H, NH₂).

### Step 1.6: 4-Chloro-6-(4-isocyanato-phenoxy)-pyrimidine

Apparatus: 18 liter reaction vessel, dropping funnel and condenser. A phosgene solution (20% in toluene, 1.43 l; 2.9 Mol) diluted with 10 of toluene under N₂-atmosphere is cooled to approximately −20° C. Then a solution of 250 g (1.12 Mol) of 4-(6-chloro-pyrimidin-4-yl-oxy)-aniline in 4.4 l of CH₂Cl₂ is added during 30 min. The resulting suspension is heated to distill off approximately 4.5 l of solvent. Distillation is continued (boiling point: 110° C.) giving a clear solution (≈3 l) in the reaction vessel, which is cooled to rt and concentrated in vacuo. Distillation of the resulting waxy crude product at 0.2 mbar gives the title compound as a solid: m.p.: 103° C.

### Example 2

### 1-[(5-tert-Butyl-2-p-tolyl-2H-pyrazol-3-yl)-3-[4-(6-methylamino-pyrimidin-4-yloxy)-phenyl]-urea

95 mg (0.2 mMol) of 1-(5-tert-butyl-2-p-tolyl-2H-pyrazol-3-yl)-3-[4-(6-chloro-pyrimidin-4-yl-oxy)-phenyl]urea (step 1.2) is dissolved in methylamine (33% in EtOH) and stirred for 14 h. After completion, the reaction mixture is concentrated, and the residual crude product is submitted to flash chromatography (SiO₂; CH₂Cl₂/MeOH 0-5% MeOH) to give the title compound as a white foam. MS: [M+1]⁺=471; ¹H-NMR (MeOH-d₄): 7.47 (d, J=9.0 Hz, 2H), 7.41 (s, 4H), 7.09 (d, J=9.0 Hz, 2H), 6.45 (s, 1H), 5.70 (s, 1H), 5.53 (s, 1H), 2.86 (s, 3H), 2.47 (s, 3H), 1.37 (s, 9H).

### Example 3

The following compounds can be obtained in analogy to Example 1 or 2 starting from commercially available phenyl hydrazines

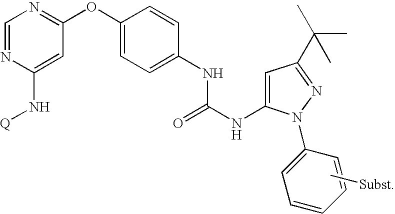

| | | | MS | HPLC | |
|---|---|---|---|---|---|
| Ex | Subst. | Q | [M + 1] | Rₜ^{A} | ¹H NMR |
| 3a | 4-isopropyl | H | 486 | | (DMSO-d₆) 9.06 (s, 1 H), 8.37 (s, 1 H), 8.03 (s, |
| | | | | | 1 H), 7.43 (d, J = 9.2 Hz, 2 H), 7.41-7.39 (m, |
| | | | | | 4 H), 7.03 (d, J = 9.2 Hz, 2 H), 6.79 (bs, 2 H), |
| | | | | | 6.35 (s, 1 H), 5.64 (s, 1 H), 4.12-4.07 (m, 1 H), |
| | | | | | 3.15 (d, J 0 5.1 Hz, 6 H), 1.26 (s, 9 H). |
| 3b | | CH₃ | | | (CDCl₃) 8.26 (s, 1 H), 7.39 (d, J = 8.2 Hz, 2 H), |
| | | | | | 7.32-7.30 (m, 4 H), 7.03 (d, J = 8.4 Hz, 2 H), |
| | | | | | 6.75 (bs, 1 H), 6.38 (s, 1 H), 5.80 (s, 1 H), 4.98 |
| | | | | | (s, 2 H), 2.98-2.94 (m, 1 H), 1.39 (s, 9 H), 1.27 (d, |
| | | | | | J = 7.1 Hz, 6 H). |
| 3c | 4-CF₃ | H | 512 | | (DMSO-d₆) 9.06 (s, 1 H), 8.53 (s, 1 H), 8.03 (s, |
| | | | | | 1 H), 7.87 (d, J = 8.8 Hz, 2 H), 7.79 (d, J = 8.8 |
| | | | | | Hz, 2 H), 7.43 (d, J = 8.8 Hz, 2 H), 7.02 (d, J = |
| | | | | | 8.8 Hz, 2 H), 6.79 (bs, 2 H, NH2), 6.40 (s, 1 H), |
| | | | | | 5.64 (s, 1 H), 1.28 (s, 9 H). |
| 3d | | CH₃ | 526 | | (CDCl₃) 8.14 (s, 1 H), 7.65 (d, J = 2.9 Hz; 4 H), |
| | | | | | 7.27 (d, J = 8.8 Hz, 2 H), 7.14 (bs, 1 H), 6.98 (d, |
| | | | | | J = 8.8 Hz, 2 H), 6.41 (s, 1 H), 5.71 (s, 1 H), 2.90 |
| | | | | | (s, 3 H), 2.91 (d, 3 H), 1.35 (s, 9 H). |
| 3e | 3-CH₃ | H | 458 | | (DMSO-d₆) 9.05 (s, 1 H, NH), 8.34 (s, 1 H, NH), |
| | | | | | 8.02 (s, 1 H), 7.42-7.35 (m, 3 H), 7.30-2.26 (m, |
| | | | | | 2 H), 7.19 (d, J = 7.3 Hz, 1 H), 7.00 (d, J = 9.2 |
| | | | | | Hz, 2 H), 6.77 (bs, 2 H, NH2), 6.33 (s, 1 H), 5.62 |
| | | | | | (s, 1 H), 2.36 (s, 3 H), 1.25 (s, 9 H). |
| 3f | | CH₃ | 472 | | (DMSO-d₆) 9.17 (s, 1 H), 8.43 (bs, 1 H), 8.07 (bs, |
| | | | | | 1 H), 7.38 (d, J = 8.8 Hz, 2 H), 7.31-7.29 (m, |
| | | | | | 3 H), 7.26-7.25 (m, 1 H), 7.18 (d, J = 7.7 Hz, |
| | | | | | 2 H), 6.99 (d, J = 8.8 Hz, 2 H), 6.32 (s, 1 H), 5.67 |
| | | | | | (s, 1 H), 2.72 (s, 3 H), 2.33 (s, 3 H), 1.25 (s, 9 H). |
| 3g | 4-F | CH₃ | 476 | | MeOH (d₄) 8.14 (s, 1 H), 7.58-7.55 (m, 2 H), 7.46 |
| | | | | | (d, J = 7.5 Hz, 2 H), 7.34-7.30 (m, 2 H), 7.09 (d, |
| | | | | | J = 7.5 Hz, 2 H), 6.45 (s, 1 H), 5.70 (s, 1 H), 2.68 |
| | | | | | (s, 3 H), 1.37 (s, 9 H). |
| 3h | 4-OCH₃ | H | 474.2 | 3.74 | |
| 3i | 4-SO₂CH₃ | H | 522.2 | 3.64 | |
| 3j | 4-SO₂CH₃ | CH₃ | 536.2 | 3.79 | |
| 3k | 4-OMEM | H | 518.2 | 3.69 | |
| 3l | 4-CO₂CH₃ | H | 502.2 | 4.08 | |
| 3m | 4-OBn | H | 550.2 | 4.61 | |
| 3n | 4-CN | H | 469.2 | 3.98 | |

### Example 4

The following compounds can be obtained in analogy to Example 1, starting from commercially available phenyl hydrazines and 3-cyclopropyl-3-oxo-propionitrile

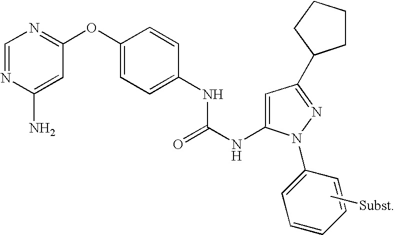

| | | MS | HPLC |
|---|---|---|---|
| Ex. | Subst. | [M + 1]⁺ | Rₜ^{A} |
| 4a | 4-CH₃ | 470.2 | 3.97 |
| 4b | 4-OCH₃ | 486.2 | 3.79 |
| 4c | 4-CN | 481.2 | 3.94 |
| 4d | 4-SO₂CH₃ | 534.2 | 3.63 |

### Example 4e

### 1-[4-(6-Amino-pyrimidin-4-yl-oxy)-phenyl]-3-(5-cyclopentyl-2-pyridin-4-yl-2H-pyrazol-3-yl)-urea

The title compound is prepared in analogy to Example 1 from commercially available 4-pyridylhydrazine and 3-cyclopropyl-3-oxo-propionitrile. MS: [M+1]⁺=457.2, Rt^{A}: 2.77 min

### Example 4f

### 1-[4-(6-Amino-pyrimidin-4-yl-oxy)-phenyl]-3-(5-phenyl-2-p-tolyl-2H-pyrazol-3-yl)-urea

The title compound is prepared in analogy to Ex. 1 from commercially available p-tolylhydrazine and benzoylacetonitrile. MS: [M+1]⁺=478.2, Rt^{A}: 4.16 min.

### Example 4g

### 1-[4-(6-Amino-pyrimidin-4-yl-oxy)-phenyl]-3-(5-furan-2-yl-2-p-tolyl-2H-pyrazol)-3-yl)-urea

The title compound is prepared in analogy to Ex. 1 from commercially available p-tolylhydrazine and 2-fuorylacetonitrile. MS: [M+1]⁺=468.2, Rt^{A}: 3.65 min.

### Example 5

### 1-[5-tert-Butyl-2-(4-morpholin-4-ylmethyl-phenyl)-2H-pyrazol-3-yl]-3-[4-(6-methylamino-pyrimidin-4-yloxy)-phenyl]-urea

101 mg (0.18 mMol) 1-[5-tert-butyl-2-(4-morpholin-4-ylmethyl-phenyl)-2H-pyrazol-3-yl]-3-[4-(6-chloro-pyrimidin-4-yloxy)-phenyl]-urea is dissolved in methylamine (33% in EtOH) and stirred at rt for 1 h. After completion of the reaction it is concentrated and the residual crude product purified by flash chromatography (SiO₂, CH₂Cl₂/MeOH gradient 0-5% MeOH) to give the title compound as a white solid. MS: [M+1]⁺=557; ¹H-NMR (CDCl₃): 8.18 (s, 1H), 7.40 (s, 4H), 7.28 (d, J=8.8 Hz, 2H), 7.01 (d, J=8.8 Hz, 2H), 6.99 (s, 1H, NH), 6.52 (s, 1H, NH), 6.36 (s, 1H), 5.72 (s, 1H), 3.71-3.68 (m, 4H), 3.50 (s, 2H), 3.06 (d, J=5.1, 3H), 2.45-2.42 (m, 4H), 1.36 (s, 9H).

### Step 5.1: 4-(5-Amino-3-tert-butyl-pyrazol-1-yl)-benzoic Acid

To a suspension of 2.4 g (16 mMol) 4-hydrazino-benzoic acid in 12 mL toluene, 2.0 g of pivaloyl acetonitrile are added at rt. The suspension is heated to and kept under reflux for 12 h. After completion, the resulting reaction mixture is allowed to cool to rt. The precipitated product is isolated by filtration, washed with cold toluene and dried under high vacuum. [M+1]⁺=260.

### Step 5.2: 5-[4-(5-Amino-3-tert-butyl-pyrazol-1-yl)-phenyl]-morpholin-4-yl-methanone

To a solution of 515 mg (1.98 mMol) 4-(5-amino-3-tert-butyl-pyrazol-1-yl)-benzoic acid and 259 μL (2.98 mMol) morpholine in 8 mL THF, 495 mg (2.58 mMol) of EDC are added at rt. The reaction is stirred at rt for 2 h. After completion, the resulting reaction mixture is concentrated and the residue is taken up in CH₂Cl₂, washed with brine (2×), dried and concentrated. The residual crude product is purified by flash chromatography (SiO₂, CH₂Cl₂/MeOH; gradient 0-5% MeOH) to give the title compound as an off white powder. MS: [M+1]⁺=329.

### Step 5.3: 5-tert-Butyl-2-(4-morpholin-4-ylmethyl-phenyl)-2H-pyrazol-3-ylamine

To a solution of 490 mg 5-[4-(5-amino-3-tert-butyl-pyrazol-1-yl)-phenyl]-morpholin-4-yl-methanone (1.49 mMol) in 13 mL THF, 3 mL (2.98 mMol) of borane (1 M solution in THF) are added at rt. The reaction is stirred at rt for 12 h, concentrated, taken up in MeOH and concentrated again (3×). The residual crude product is purified by flash chromatography (SiO₂, CH₂Cl₂/MeOH, gradient 0-5% MeOH) to give the title compound as a yellow solid. ¹H-NMR (CDCl₃): 7.50 (d, J=7.2 Hz, 2H), 7.40 (d, J=7.2 Hz, 2H), 5.52 (s, 1H), 3.73-3.70 (m, 5H), 3.51 (s, 2H), 2.47-2.44 (m, 3H), 1.32 (s, 9H).

### Step 5.4: 1-[5-tert-Butyl-2-(4-morpholin-4-ylmethyl-phenyl)-2H-pyrazol-3-yl]-3-[4-(6-chloropyrimidin-4-yloxy)-phenyl]-urea

To a solution of 308 mg (0.97 mMol) 5-tert-butyl-2-(4-morpholin-4-ylmethyl-phenyl)-2H-pyrazol-3-ylamine in 9 mL ether, a solution of 243 mg (0.97 mMol) 4-chloro-6-(4-isocyanato-phenoxy)-pyrimidine (step 1.6) is added in 3 mL THF at rt. The reaction is stirred for 24 h at rt and then concentrated. The crude product is, purified by flash chromatography (SiO₂, CH₂Cl₂/MeOH, gradient 0-5% MeOH) to give the title compound as a white foam. MS: [M+1]⁺=563.

### Example 6

### 1-[4-(6-Amino-pyrimidin-yloxy)-phenyl]-3-[5-ter-butyl-2-(4-morpholin-4-ylmethyl-phenyl)-2H-pyrazol-3-yl]-urea

A solution of 105 mg (0.18 mMol) 1-[4-(6-azido-pyrimidin-4-yloxy)-phenyl]-3-[5-tert-butyl-2-(4-morpholin-4-ylmethyl-phenyl)-2H-pyrazol-3-yl]-urea in 5 mL MeOH is hydrogenated over Pd/C (10% Engelhardt 4045) at rt under atmospheric pressure for 2 h. After completion of the reaction, the catalyst is filtered off and the filtrate is concentrated. The residual crude product is purified by flash chromatography (SiO₂, CH₂Cl₂/MeOH, gradient 0-8% MeOH) to give the title compound as a yellow solid. MS: [M+1]⁺=543; ¹H-NMR (CDCl₃): 8.21 (s, 1H), 7.40 (s, 4H), 7.28 (d, J=8.8 Hz, 2H), 7.02 (s, 1H, NH), 7.00 (d, J=8.8 Hz, 2H), 6.59 (s, 1H, NH), 6.35 (s, 1H), 5.77 (s, 1H), 5.30 (s, 1H), 4.94 (s, 2H, NH₂), 3.70-3.67 (m, 4H), 3.50 (s, 2H), 2.45-2.42 (m, 4H), 1.36 (s, 9H).

### Step 6.1: 1-[4-(6-Azido-pyrimidin-4-yloxy)-phenyl]-3-[5-tert-butyl-2-(4-morpholin-4-ylmethyl-phenyl)-2H-pyrazol-3yl]-urea

A solution of 117 mg (0.21 mMol) 1-[5-tert-butyl-2-(4-morpholin-4-ylmethyl-phenyl)-2H-pyrazol-3-yl]-3-[4-(6-chloro-pyrimidin-4-yloxy)-phenyl]-urea in 3 mL DMF is treated with 27 mg (0.42 mMol) sodium azide at rt. The reaction mixture is them warmed to 70° C. for 2.5 h. It is allowed to cool to rt again and concentrated. The residue is taken up in EtOAc and washed with brine (2×). The organic layer is tried and concentrated to give the crude title compound which is used without further purification for the next step. MS: [M+1]⁺=569.

### Example 7

The following compounds can be obtained in analogy to Example 5 or 6 starting from either 3- or 4-hydrazino-benzoic acid

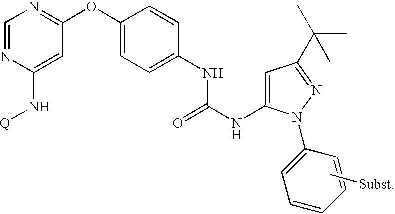

| | | | MS | |
|---|---|---|---|---|
| Ex. | Subst. | Q | [M + 1]⁺ | ¹H NMR |
| 7a | 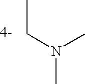 | H | 500 | (MeOH-d₄) 8.07 (s, 1 H), 7.50 (s, 4 H), 7.43 (d, J = 10.4 Hz, 2 H), 7.06 (d, J = 10.4 Hz, 2 H), 6.42 (s, 1 H), 5.73 (s, 1 H), 3.57 (s, 2 H), 2.29 (s, 6 H), 1.35 (s, 9 H). |
| 7b | | CH₃ | 515 | (MeOH-d₄) 8.10 (s, 1 H), 7.50 (s, 4 H), 7.43 (d, J = |
| | | | | 9.1 Hz, 2 H), 7.05 (d, J = 9.1 Hz, 2 H), 6.42 (s, |
| | | | | 1 H), 3.58 (s, 3 H), 2.84 (s, 2 H), 2.30 (s, 6 H), 1.35 |
| | | | | (s, 9 H). |
| 7c | 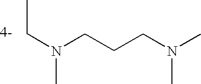 | CH₃ | 586 | (CDCl₃) 8.20 (s, 1 H), 7.44-7.38 (m, 6 H), 7.00 (d, J = 8.8 Hz, 2 H), 6.95 (s, 1 H, NH), 6.46 (s, 1 H), 5.66 (s, 1 H), 5.30 (s, 1 H), 5.11 (s, 1 H, NH), 3.48 (s, 2 H), 2.90 (d, J = 5.1 Hz, 3 H), 2.64-2.59 (m, 2 H), 2.49 (s, 6 H), 2.38 (t, J = 6.2 Hz, 2 H), 2.33 (s, 3 H), 1.73-1.63 (m, 2 H), 1.35 (s, 9 H). |
| 7d | 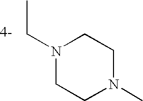 | H | 501 | (MeOH-d₄) 8.06 (s, 1 H), 7.49 (d, J = 7.4 Hz, 4 H), 7.42 (d, J = 7.0 Hz; 2 H); 7.04 (d, J = 7.0 Hz, 2 H), 6.40 (s, 1 H), 3.62 (s, 3 H), 2.64-2.48 (m, 8 H), 2.39 (s, 3 H , 1.34 (s, 9 H). |
| 7e | | CH₃ | 570 | (CDCl₃) 8.10 (s, 1 H), 7.85 (s, 1 H), 7.32 (d, J = |
| | | | | 7.4 Hz, 2 H), 7.28 (d, J = 7.4 Hz; 2 H), 7.20 (d, J = |
| | | | | 7.21 Hz, 2 H), 6.90 (d, J = 7.1 Hz, 2 H), 6.35 (s, |
| | | | | 1 H), 5.60 (s, 1 H), 5.43-5.41 (m, 1 H, NH), 3.44 (s, |
| | | | | 2 H), 2.87 (d, J = 5.4 Hz, 3 H), 2.50-2.34 (m, 8 H), |
| | | | | 2.25 (s, 3 H), 1.32 (s, 9 H). |
| 7f | 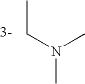 | H | 501 | (MeOH-d₄) 8.06 (s, 1 H), 7.54-7.50 (m, 5 H), 7.43 (d, J = 7.4 Hz, 2 H), 7.04 (d, J = 7.4 Hz, 2 H), 6.42 (s, 1 H), 5.73 (s, l H), 3.63 (s, 2 H), 2.36 (s, 6 H), 1.35 (s, 9 H). |
| 7g | | CH₃ | 515 | (CDCl₃) 8.33, (s, 1 H, NH), 8.15 (s, 1 H), 7.79 (s, |
| | | | | 1 H, NH), 7.40-7.25 (m, 5 H), 7.15 (d, J = 8.6 Hz, |
| | | | | 1 H), 6.93 (d, J = 10.1 Hz, 2 H), 6.43 (s, 1 H), 5.57 |
| | | | | (s, 1 H), 5.83 (s, 1 H, NH), 3.41 (s, 2 H), 2.86 (d, J = |
| | | | | 6.2 Hz, 3 H), 2.25 (s, 6 H), 1.33 (s, 9 H). |
| 7h | 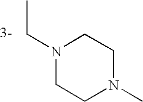 | H | 555.8 (neg) | (MeOH-d₄) 8.07 (s, 1 H), 7,50-7.46 (m, 1 H), 7.45- 7.42 (m, 4 H), 7.05 (d, 2 H), 6.40 (s, 1 H), 5.73 (s, 1 H), 3.62 (s, 2 H), 2.56-2.47 (m, 8 H), 2.27 (s, 3 H), 1.35 (s, 9 H). |
| 7i | | CH₃ | 570 | (MeOH-d₄) 8.10 (s, 1 H), 7.51-7.41 (m, 6 H), 7.05 |
| | | | | (d, J = 9.0 Hz, 2 H), 6.40 (s, 1 H), 5.65 (s, 1 H), |
| | | | | 3.66-3.59 (m, 6 H), 3.61 (s, 2 H), 2.83 (s, 3 H), |
| | | | | 2.51-2.49 (m, 4 H), 1.35 (s, 9 H). |
| 7j | 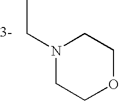 | H | 542 (neg) | (MeOH-d₄) 8.07 (s, 1 H), 7.52-7.50 (m, 2 H), 7.43 (d, 4 H), 7.04 (d, 2 H), 6.40 (s, 1 H), 5.71 (s, 1 H), 3.66-3.61 (m, 4 H), 3.31 (s, 2 H), 2.53-2.50 (m, 4 H), 1.35 (s, 9 H). |
| 7k | | CH₃ | 562 | (MeOH-d₄) 8.10 (s, 1 H), 7.51-7.49 (m, 2 H), 7.42 |
| | | | | (d, 4 H), 7.04 (d, 2 H), 6.40 (s, 1 H), 5.65 (s, 1 H), |
| | | | | 3.66-3.59 (m, 4 H), 3.61 (s, 2 H), 2.83 (s, 3 H), |
| | | | | 2.51-2.48 (m, 4 H), 1.35 (s, 9 H). |
| 7l | 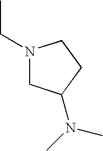 | H | 569 (neg) | (MeOH-d₄) 8.07 (s, 1 H), 7.50 (d, J = 9.3 Hz, 4 H), 7.43 (d, J = 8.5 Hz, 2 H), 7.06 (d, J = 8.5 Hz, 2 H), 6.42 (s, 1 H), 5.73 (s, 1 H), 3.75 (d, J = 12.7 Hz, 1 H), 3.67 (d, J = 12.7 Hz, 1 H), 2.93-2.74 (m, 3 H), 2.62-2.53 )m, 1 H), 2.39-2.35 (m, 1 H), 2.23 (s, 6 H), 2.09-1.96 (m, 1 H), 1.81-1.71 (m, 1 H), 1.34 (s, 9 H). |
| 7m | | CH₃ | 584 | (MeOH-d₄) 8.10 (s, 1 H), 7.52 (s, 4 H), 7.43 (d, J = |
| | | | | 7.4 Hz, 2 H), 7.04 (d, J = 7.4 Hz, 2 H), 6.40 (s, |
| | | | | 1 H), 5.68 (s, 1 H), 3.88-3.72 (m, 3 H), 3.09-2.94 |
| | | | | (m, 2 H), 2.81 (s, 9 H), 2.64-2.53 (m, 1 H), 2.39- |
| | | | | 2.30 (m, 1 H), 2.11-1.97 (m, 1 H), 1.35 (s, 1 H). |

### Example 8

### 1-[5-tert-Butyl-2-(4-fluoro-phenyl)-2H-pyrazol-3-yl]-3-(4-{6-[2-(1-methyl-pyrrolidin-2-yl)-ethylamino]-pyrimidin-4-yloxy}phenyl)-urea

To a solution of 1-[5-tert-butyl-2-(4-fluoro-phenyl)-2H-pyrazol-3-yl]-3-[4-(6-chloro-pyrimidin-4-yloxy)-phenyl]urea (30 mg, 0.062 mMol) in DMF is added 2-(2-aminomethyl)-1-methyl pyrrolidine (52 mL, 0.37 mMol) and the reaction mixture is stirred at 70° C. for 5 h. After cooling and removal of all volatiles the crude product is purified by HPLC Rt^{B}: 2.38 min; MS: [M+1]⁺=573.1

### Step 8.1: 1-(5-tert-Butyl-2-(4-fluorophenyl)-2H-pyrazol-3-yl)-3-[4-(6-chloro-pyrimidin-4-yloxy)-phenyl]urea

A solution of 200 mg (0.87 mMol) of 5-tert-butyl-(4-fluorophenyl)-2H-pyrazol-3-yl amine in 9 mL of ether is treated with a solution of 211 mg (0.87 mMol) 4-chloro-6-(4-isocyanato-phenoxy)-pyrimidine (see step 1.6) in 3 mL THF at rt. The reaction is stirred for 2.5 h at rt and then warmed to and kept at 40° C. for 12 h. After completion, the reaction mixture is concentrated in vacuo, and the resulting crude product is purified by flash chromatography (SiO₂; MeOH/CH₂Cl₂; gradient 0-5% MeOH) to give the title compound as a white foam. MS: [M+1]⁺=478.

The following examples were prepared according to the procedure described for Ex. 7 with the appropriate amines

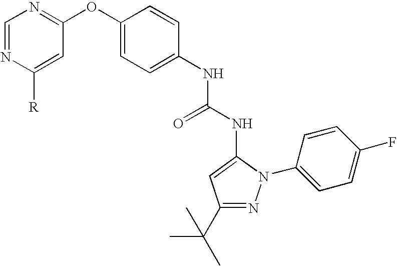

| | | MS | HPLC |
|---|---|---|---|
| Ex | R | [M + 1]⁺ | Rₜ^{B} |
| 8a | 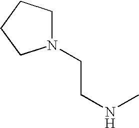 | 559.1 | 2.47 |
| 8b | 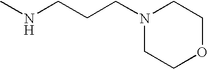 | 589.1 | 2.38 |
| 8c | 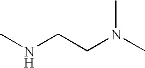 | 533.1 | 2.37 |
| 8d | 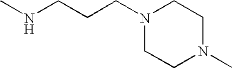 | 602.16 | 2.37 |
| 8e | 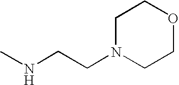 | 575.11 | 2.52 |
| 8f | 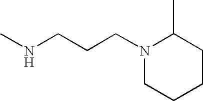 | 601.13 | 2.62 |
| 8g | 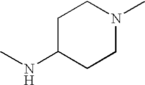 | 559.1 | 2.32 |

added to the orange suspension and the resulting reaction mixture stirred at 65° C. overnight. The reaction mixture is then cooled to 0° C. and the precipitated product isolated by filtration, washed with cold H₂O/acetone (1:1) and dried at high vacuum. ¹H-NMR (CDCl₃): 8.65 (s, 1H), 6.90 (d. 1H), 6.85 (s, 1H), 6.33 (s, 1H), 6.28 (d, 1H), 3.75 (bs, 5H, NH₂/OCH₃).

### Step 11.2: 4-(6-Chloro-pyrimidin-4-yloxy)-3-methoxy-phenylamine

4-Chloro-6-(2-methoxy-4-nitro-phenoxy)-pyrimidine (430 mg, 1.5 mMol) is dissolved in THF/MeOH (1:1; 8 mL) and submitted to hydrogenation over Raney-Nickel at ambient pressure and temperature for 14 h. After completion of the reaction it is filtered over a pad of celite, concentrated and dried to give the title compound. MS: [M+1]⁺=252.0; R_{f} (CH₂Cl₂/MeOH 90:10) 0.67.

### Step 11.3: 5-tert-Butyl-2-m-tolyl-2H-pyrazol-3-ylamine

m-Tolylhydrazine (3.81 g, 31 mMol) and pivaloylacetonitrile (3.91 g 31 mMol) are dissolved in toluene (30 mL) and refluxed for 12 h. After cooling and removal of all volatiles the crude product is purified by flash chromatography (SiO₂, 100% CH₂Cl₂) to give the title compound. MS: [M+1]⁺=230.11; ¹H-NMR (CDCl₃): 7.39 (s, 1H), 7.37 (m, 2H), 7.15 (m, 1H), 5.43 (s, 1H), 3.71 (bs, 2H, NH₂), 2.39 (s, 3H), 1.34 (s, 9H).

### Step 11.4: 1-(5-tert-Butyl-2-m-tolyl-2H-pyrazol-3-yl)-3-[4-(6-chloro-pyrimidin-4-yloxy)-3-methoxy-phenyl]-urea

4-(6-Chloro-pyrimidin-4-yloxy)-3-methoxy-phenylamine (280 mg, 1.1 mMol) is dissolved in a 20 wt % solution of phosgene in toluene (4 mL) and heated to reflux for 1 h. The reaction is then allowed to cool and all volatiles are removed in vacuo. The remaining crude isocyanate is treated with a solution of 5-tert-butyl-2-m-tolyl-2H-pyrazol-3-ylamine (255 mg, 1.1 mMol) in THF (5 mL) at rt. The reaction is stirred for 13 h and then concentrated. The remaining crude product is purified by flash chromatography (SiO₂; CH₂Cl₂/MeOH, gradient 0-5% MeOH) to give the title compound. MS: [M+1]⁺=508.79; R_{f} (CH₂Cl₂/MeOH 95:5) 0.3.

### Example 12

The following compounds can be obtained in analogy to Example 11 starting from the appropriate 2-amino-pyrazoles and 4-(6-chloro-pyrimidin-4-yloxy)-3-methoxy-phenylamine (step 13.2) by treatment of the intermediate 6-chloro-pyrimidine ureas (analogous to step 13.4) either according to the procedure described for example 11 or example 1 (step 1.3)

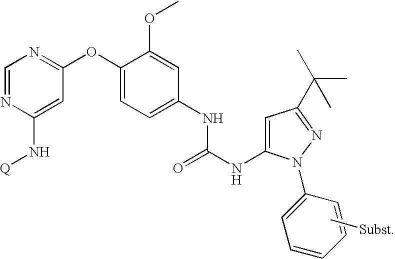

| | | | MS | |
|---|---|---|---|---|
| Ex | Subst | Q | [M + 1]⁺ | Mp [° C.] |
| 12a | 4-iso-propyl | CH₃ | 529.96 | 129-131 |
| 12b | 4-SO₂CH₃ | H | 552.96 | 161-163 |
| 12c | 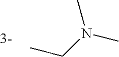 | CH₃ | 544.96 | 117-119 |
| 12d | 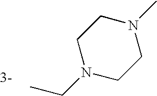 | CH₃ | 600.96 | 134-135 |
| 12e | 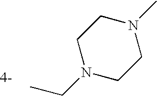 | CH₃ | 600.04 | 125-127 |
| 12f | 4-OCH₃ | H | 504.10 | 148-150 |
| 12g | 4-F | CH₃ | 505.90 | 136-138 |

### Example 13

### 1-[4-(6-Amino-pyrimidin yloxy)-3-fluoro-phenyl]-3-[5-tert-butyl-2-(4-methoxy-phenyl]-2H-pyrazol-3-yl]-urea

Prepared in analogy to Example 1 from 1-[4-(6-azido-pyrimidin-4-yloxy)-3-fluoro-phenyl]-3-1-[5-tert-butyl-2-(4-methoxy-phenyl)-2H-pyrazol-3-yl]-urea. M.p. 185-186 DC; ¹H-NMR (DMSO-d₆): 8.33 (s, 1H), 8.00 (s, 1H), 7.52 (d, 1H), 7.39 (d, 2H), 7.16 (dd, 1H), 7.06 (s, 1H), 7.04 (d, 2H), 6.85 (bs, 1H), 6.31 (s, 1H), 5.77 (s, 1H), 3.79 (s, 3H), 1.25 (s, 9H).

### Step 13.1: 4-Chloro-6-(2-fluoro-4-nitro-phenoxy)-pyrimidine

Prepared in analogy to step 11.1 from 4,6-dichloro pyrimidine and 2-fluoro-4-nitro phenol MS: [M+1]⁺=270.27; ¹H-NMR (CDCl₃): 8.55 (s, 1H), 8.26-8.18 (m, 2H), 7.65 (d, 1H), 7.43 (s, 1H).

### Step 13.2: 4-(6-Chloro-pyrimidin-4-yloxy)-3-fluoro-phenylamine

Prepared in analogy to step 11.2 from 4-chloro-6-(2-fluoro-4-nitro-phenoxy)-pyrimidine. MS: [M+1]⁺=240.32.

### Step 13.3: 5-tert-Butyl-(4-methoxy phenyl)-2H-pyrazol-3-ylamine

Prepared in analogy to step 11.3 from 4-methoxy phenyl hydrazine. MS: [M+1]⁺=246.41; ¹H-NMR (CDCl₃): 7.41 (d, 2H), 6.97 (d, 2H), 5.43 is, 1H), 3.83 (s, 3H), 1.35 (s, 9H).

### Step 13.4: 1-(5-ter-Butyl-2-(4-methoxy-phenyl)-2H-pyrazol-3-yl]-3-[4-(6-chloro-pyrimidin-4yloxy)-3-fluoro-phenyl]-urea

Prepared in analogy to step 11.4 from 4-(6-chloro-pyrimidin-4-yloxy)-3-fluoro-phenylamine and 5-tert-Butyl-(4-methoxy-phenyl)-2H-pyrazol-3-ylamine. MS: [M+1]⁺=511.38, ¹H-NMR (CDCl₃): 8.53 (s, 1H), 7.46 (d, 1H), 7.37-7.35 (m, 2H), 7.24-7.22 (m, 1H), 7.11-7.09 (m, 1H), 7.00 (s, 1H), 6.92 (d, 2H), 6.44 (bs, 1H, NH), 6.32 (s, 1H, NH), 5.50 (s, 1H), 3.82 (s, 3H), 1.36 (s, 9H).

### Step 13.5: 1-[4-(6-Azido-pyrimidin-4-yloxy)-3-fluoro-phenyl]-3-[5-tert-butyl-2-(4-methoxy-phenyl)-2H-pyrazol-3-yl]-urea

Prepared in analogy to step 1.3 from 1-(5-tert-butyl-2-(4-methoxy-phenyl)-2H-pyrazol-3-yl]-3-[4-(6-chloro-pyrimidin-4-yloxy)-3-fluoro-phenyl]-urea. MS: [M+1]⁺=518.43.

### Example 14

### 1-[4-(6-Amino-pyrimidin-4-yloxy)-2-fluoro-phenyl]-3-[5-tert-butyl-2-(4-fluoro-phenyl]-2H-pyrazol-3-yl]-urea

Prepared in analogy to example 1 from 1-[4-(6-azido-pyrimidin-4-yloxy)-2-fluoro-phenyl]-3-1-[5-tert-butyl-2-(4-fluoro-phenyl)-2H-pyrazol-3-yl]-urea. Mp 141-142° C.; MS: [M+1]⁺ 480.41.

### Step 14.1: 4-Chloro-6-(3-fluoro-4-nitro-phenoxy)-pyrimidine

Prepared in analogy to step 11.1 from 4,6-dichloro pyrimidine and 3-fluoro-4-nitro phenol MS: [M+1]⁺=270.05; ¹H-NMR (MeOH-d₄): 8.60 (s, 1H), 8.23 (dd, 1H), 7.45 (d, 1H), 7.35 (s, 1H), 7.27 (dd, 1H).

### Step 14.2: 4-(6-Chloro-pyrimidin-4-yloxy)-2-fluoro-phenylamine

Prepared in analogy to step 11.2 from 4-chloro-6-(3-fluoro-4-nitro-phenoxy)-pyrimidine. MS: [M+1]⁺=240.28; ¹H-NMR (MeOH-d₄): 8.53 (s, 1H), 7.04 (s, 1H), 6.91-6.85 (m, 2H), 6.76 (d, 1H).

### Step 14.3: 5-tert-Butyl-(4-fluoro phenyl)-2H-pyrazol-3-ylamine

Prepared in analogy to step 11.3 from 4-fluoro; phenyl hydrazine. MS: [M+1]⁺=246.41, ¹H-NMR (CDCl₃): 7.56 (d, 2H), 7.19 (d, 2H), 5.53 (s, 1H), 3.63 (bs, 2H, NH2), 1.35 (s, 9H).

### Step 14.4: 1-(5-ter-Butyl-2-(4-fluoro-phenyl)-2H-pyrazol-3-yl]-3-[4-(6-chloro-pyrimidin-4yloxy)-2-fluoro-phenyl]-urea

Prepared in analogy to step 11.4 from 4-(6-chloro-pyrimidin-4-yloxy)-2-fluoro-phenylamine and 5-tert-butyl-(4-fluoro phenyl)-2H-pyrazol-3-ylamine. MS: [M+1]⁺ 499.29, ¹H-NMR (CDCl₃): 8.56 (s, 1H), 8.15 (d, 1H), 7.44 (dd, 2H), 7.12 (dd, 2H), 6.94 (bs, 1H, NM, 6.92 (d, 4H), 6.70 (sb, 1H, NH), 6.37 (s, 1H), 1.36 (s, 9H).

### Step 14.5: 1-[4-(6-Azido-pyrimidin-4-yloxy)-2-fluoro-phenyl]-3-1-[5-tert-butyl-2-(4-fluoro-phenyl)-2H-pyrazol-3-yl]-urea

Prepared in analogy to step 1.3 from 1-(5-tert-butyl-2-(4-fluoro-phenyl)-2H-pyrazol-3-yl]-3-[4-(6-chloro-pyrimidin-4-yloxy)-2-fluoro-phenyl]-urea. MS: [M+1]⁺=506.33. ¹H-NMR (CDCl₃): 8.53 (s, 1H), 8.21 (d, 1H), 7.82 (s, 1H), 7.79 (bs, 1H), 7.46 (dd, 2H), 7.11 (dd, 2H), 6.93-6.87 (m, 2H), 6.42 (s, 1H), 6.28 (s, 1H), 1.34 (s, 9H).

The following examples can be prepared in analogy to the procedures described above:

### Example 15

### 1-{5-tert-Butyl-2-[3-(4-methyl-piperazine-1-carbonyl)-phenyl]-2H-pyrazol-3-yl}-3-[4-(6-methylamino-pyrimidin-4-yloxy)-phenyl]-urea

M.p. 146-147° C.; MS: [M+1]⁺ 585.

### Example 16

### 1-[4-(6-Amino-pyrimidin-4-yloxy)-phenyl]-3-{5-tert-butyl-2-[4-(morpholine-4-carbonyl)-phenyl]-2H-pyrazol-3-yl}-urea

M.p. 160-161° C.; MS: [M+1]⁺ 558.

### Example 17

### 1-{5-tert-Butyl-2-[4-(morpholine-4-carbonyl)-phenyl]-2H-pyrazol-3-yl}-3-[4-(6-methylamino-pyrimidin-4-yloxy)-phenyl]-urea

M.p. 148-149° C.; MS: [M+1]+572.

### Example 18

### Soft Capsules

5000 soft gelatin capsules, each comprising as active ingredient 0.05 g of one of the compounds of formula I mentioned in any one of the preceding examples, are prepared as follows:

Composition

| | | |
|---|---|---|
| Active ingredient | 250 | g |
| Lauroglycol | 2 | litres |

Preparation process: The pulverized active ingredient is suspended in Lauroglykol* (propylene glycol laurate, GattefosséS. A., Saint Priest, France) and ground in a wet pulverizer to produce a particle size of about 1 to 3 μm. 0.419 g portions of the mixture are then introduced into soft gelatin capsules using a capsule-filling machine.

### Example 19

### Tablets Comprising Compounds of the Formula I

Tablets, comprising, as active ingredient, 100 mg of any one of the compounds of formula I of Examples 1 to 7 are prepared with the following composition, following standard procedures:

Composition

| | | |
|---|---|---|
| Active Ingredient | 100 | mg |
| crystalline lactose | 240 | mg |
| Avicel | 80 | mg |
| PVPPXL | 20 | mg |
| Aerosil | 2 | mg |
| magnesium stearate | 5 | mg |
| | 447 | mg |

Manufacture: The active ingredient is mixed with the carrier materials and compressed by means of a tabletting machine (Korsch EKO, Stempeldurchmesser 10 mm).

Avicel® is microcrystalline cellulose (FMC, Philadelphia, USA). PVPPXL is polyvinylpolypyrrolidone, cross-linked (BASF, Germany). Aerosil® is silicium dioxide (Degussa, Germany).

We claim:

1. A compound of the formula I, 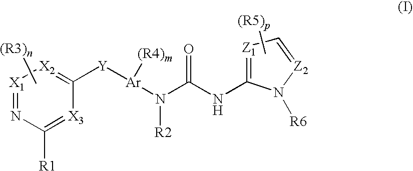
wherein
R1 is hydrogen, or unsubstituted or substituted alkyl, halogen, hydroxy, esterified or etherified hydroxy, amino, substituted amino, carboxy, esterified carboxy, carbamoyl, N-mono- or N,N-disubstituted carbamoyl;
R2 is unsubstituted or substituted alkyl, unsubstituted or substituted aryl, unsubstituted or substituted heterocyclyl or unsubstituted or substituted cycloalkyl,
n is 0, 1, 2 or 3;
m is 0, 1, 2 or 3;
p is 0, 1, 2 or 3;
each of R3 and R4, if present and independents of the others, is unsubstituted or substituted alkyl, halogen, hydroxy, esterified or etherified hydroxy, mercapto, substituted mercapto, nitro, amino, substituted amino, carboxy, esterified carboxy, carbamoyl, N-mono- or N,N-disubstituted carbamoyl, sulfo, esterified sulfo, sulfamoyl, N-mono- or N,N-disubstituted sulfamoyl or cyano;
R5, independently of R3 and R4, is unsubstituted or substituted alkyl, unsubstituted or substituted aryl, unsubstituted or substituted heterocyclyl or unsubstituted or substituted cycloalkyl, halogen, hydroxy, esterified or etherified hydroxy, mercapto, substituted mercapto, nitro, amino, substituted amino, carboxy, esterified carboxy, carbamoyl, N-mono- or N,N-disubstituted carbamoyl, sulfo, esterified sulfo, sulfamoyl, N-mono- or N,N-disubstituted sulfamoyl or cyano;
R6 is unsubstituted or substituted alkyl, unsubstituted or substituted aryl, unsubstituted or substituted heterocyclyl or unsubstituted or substituted cycloalkyl;
each of X₁, X₂ and X₃, independently of the others, is N or CH;
Y is oxy (—O—), imino (—NH—), thio (—S—) or methylene (—CH₂—) and
Ar is arylene or heterocyclylene;
and each of Z₁ and Z₂, independently of the other, is nitrogen (N) or CH, with the proviso that at least one of Z₁ and Z₂ is N;
or a salt thereof.

2. A compound of the formula I according to claim 1, wherein in each case of occurrence in claim 1
unsubstituted or substituted alkyl is C₁-C₂₀-alkyl, more preferably C₁-C₇-alkyl, that is straight-chained or branched (one or, if desired and possible, more times), and which is unsubstituted or substituted by one or more, e.g. up to three moieties selected from unsubstituted or substituted aryl as described below, especially phenyl or naphthyl, which is unsubstituted or substituted as described below for unsubstituted or substituted aryl, unsubstituted or substituted heterocyclyl as described below which is unsubstituted or substituted as described below for unsubstituted or substituted heterocyclyl, especially piperidino, morpholino, thio-morpholino, N—C₁-C₇-alkyl-piperazino, or N-mono- or N,N-di-(C₁-C₇-alkyl-substituted or unsubstituted pyrrolidino, unsubstituted or substituted cycloalkyl as described below, especially cyclopropyl, cyclobutyl, cyclopentyl or cyclohexyl each of which is unsubstituted or substituted as described below for unsubstituted or substituted cycloalkyl, halo, e.g. in trifluoro-methyl, hydroxy, C₁-C₇-alkoxy, halo-C₁-C₇-alkoxy, such as trifluoromethoxy, hydroxy-C₁-C₇-alkoxy, C₁-C₇-alkoxy-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl and/or C₁-C₇-alkoxy-C₁-C₇alkyl and/or mono or di-(C₁-C₇-alkyl)-amino-C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl)-aminocarbonyl, N-mono- or N,N-di-(naphthyl- or phenyl-C₁-C₇-alkyl)-aminocarbonyl, cyano, C₁-C₇-alkenylene or -alkynylene, C₁-C₇-alkylenedioxy, sulfeno, O—C₁-C₇-alkylsulfenyl, O-phenyl- or naphthylsulfenyl wherein phenyl or naphthyl is unsubstituted or substituted by one or more, especially one to three, C₁-C₇-alkyl moieties, O-phenyl- or naphthyl-C₁-C₇-alkylsulfenyl, sulfino, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfo, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl, naphthyl, phenyl-C₁-C₇-alkyl or naphthyl-C₁-C₇-alkyl)-aminosulfonyl;
unsubstituted or substituted aryl is a mono- or polycyclic, especially monocyclic, bicyclic or tricyclic aryl moiety with 6 to 22 ring carbon atoms, especially phenyl, naphthyl, indenyl, fluorenyl, acenapthylenyl, phenylenyl or phenanthryl, and is unsubstituted or substituted by one or more, especially one to three, moieties, preferably independently selected from the group consisting of C₁-C₇-alkyl, such as methyl, ethyl, n-propyl, isopropyl, n-butyl, isobutyl, sec-butyl or tert-butyl, C₂-C₇-alkenyl, C₂-C₇-alkinyl, phenyl- or naphthyl-C₁-C₇-alkyl, such as benzyl or naphthylmethyl, halo-C₁-C₇-alkyl, such as trifluoromethyl, hydroxy-C₁-C₇-alkyl, C₁-C₇-alkoxy-C₁-C₇-alkyl, such as 3-methoxypropyl or 2-methoxyethyl, C₁-C₇-alkoxy-C₁-C₇-alkoxy-C₁-C₇-alkyl, phenyloxy- or naphthyloxy-C₁-C₇-alkyl, phenyl-C₁-C₇-alkoxy- or naphthyl-C₁-C₇-alkoxy-C₁-C₇-alkyl, amino-C₁-C₇-alkyl, such as aminomethyl, N-mono- or N,N-di-(C₁-C₇-alkyl and/or mono-C₁-C₇-alkoxy-C₁-C₇alkyl and/or (mono- or di-(C₁-C₇-alkyl)-amino-C₁-C₇-alkyl)-amino-C₁-C₇-alkyl, C₁-C₇-alkoxy-C₁-C₇-alkylamino-C₁-C₇-alkyl, mono- or di-(naphthyl- or phenyl-C₁-C₇-alkyl)-amino-C₁-C₇-alkyl, C₁-C₇-alkanoylamino-C₁-C₇-alkyl, carboxy-C₁-C₇-alkyl, benzoyl- or naphthoylamino-C₁-C₇-alkyl, C₁-C₇-alkylsulfonylamino-C₁-C₇-alkyl, phenyl- or naphthylsulfonylamino-C₁-C₇-alkyl wherein phenyl or naphthyl is unsubstituted or substituted by one or more, especially one to three, C₁-C₇-alkyl moieties, phenyl- or naphthyl-C₁-C₇-alkylsulfonylamino-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, N-mono- or N,N-di-(C₁-C₇-alkyl)-amino-substituted or unsubstituted pyrrolidino-C₁-C₇-alkyl, halo, especially fluoro, chloro or bromo, hydroxy, C₁-C₇-alkoxy, phenyl-C₁-C₇-alkoxy wherein phenyl is unsubstituted or substituted by C₁-C₇-alkoxy and/or halo, halo-C₁-C₇-alkoxy, such as trifluoromethoxy, hydroxy-C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, N-unsubstituted-, N-mono- or N,N-di-(C₁-C₇-alkylcarbamoyl-C₁-C₇-alkoxy, phenyl- or naphthyloxy, phenyl- or naphthyl-C₁-C₇-alkyloxy, C₁-C₇-alkanoyloxy, benzoyl- or naphthoyloxy, C₁-C₇-alkylthio, halo-C₁-C₇-alkthio, such as trifluoromethylthio, C₁-C₇-alkoxy-C₁-C₇-alkylthio, phenyl- or naphthylthio, phenyl- or naphthyl-C₁-C₇-alkylthio, C₁-C₇-alkanoylthio, benzoyl- or naphthoylthio, nitro, amino, mono- or di-(C₁-C₇-alkyl)-amino, mono- or di-(naphthyl- or phenyl-C₁-C₇-alkyl)-amino, C₁-C₇-alkanoylamino, benzoyl- or naphthoylamino, C₁-C₇-alkylsulfonylamino, phenyl- or naphthylsulfonylamino wherein phenyl or naphthyl is unsubstituted or substituted by one or more, especially one to three, C₁-C₇-alkyl moieties, phenyl- or naphthyl-C₁-C₇-alkylsulfonylamino, C₁-C₇-alkanoyl, C₁-C₇-alkoxy-C₁-C₇-alkanoyl, carboxyl, C₁-C₇-alkyl-carbonyl, C₁-C₇-alkoxy-carbonyl, phenyl- or naphthyloxycarbonyl, phenyl- or naphthyl-C₁-C₇-alkoxycarbonyl, carbamoyl, N-mono- or N,N-di-(C₁-C₇-alkyl and/or mono-C₁-C₇-alkoxy-C₁-C₇alkyl and/or (mono- or di-(C₁-C₇-alkyl)-amino-C₁-C₇-alkyl)-amino-carbonyl, such as N-mono- or N,N-di-(C₁-C₇-alkyl)-aminocarbonyl, N—C₁-C₇-alkoxy-C₁-C₇-alkylcarbamoyl, N-mono- or N,N-di-(naphthyl- or phenyl-C₁-C₇-alkyl)-aminocarbonyl, pyrrolidinocarbonyl, piperidinocarbonyl, morpholinocarbonyl, thiomorpholinocarbonyl, N—C₁-C₇-alkyl-piperazinocarbonyl, N-mono- or N,N-di-(C₁-C₇-alkyl)-amino-substituted or unsubstituted pyrrolidino-C₁-C₇-alkyl, cyano, C₁-C₇-alkenylene or -alkinylene, C₁-C₇-alkylenedioxy, sulfeno, O—C₁-C₇-alkylsulfenyl, O-phenyl- or naphthylsulfenyl wherein phenyl or naphthyl is unsubstituted or substituted by one or more, especially one to three, C₁-C₇-alkyl moieties, O-phenyl- or naphthyl-C₁-C₇-alkylsulfenyl, sulfino, C₁-C₇-alkylsulfinyl, phenyl- or naphthylsulfinyl wherein phenyl or naphthyl is unsubstituted or substituted by one or more, especially one to three, C₁-C₇-alkyl moieties, phenyl- or naphthyl-C₁-C₇-alkylsulfinyl, sulfo, C₁-C₇-alkylsulfonyl, phenyl- or naphthylsulfonyl wherein phenyl or naphthyl is unsubstituted or substituted by one or more, especially one to three, C₁-C₇-alkyl moieties, phenyl- or naphthyl-C₁-C₇-alkylsulfonyl, sulfamoyl and N-mono or N,N-di-(C₁-C₇-alkyl, phenyl-, naphthyl-, phenyl-C₁-C₇-alkyl- or naphthyl-C₁-C₇-alkyl)-aminosulfonyl, piperidino, morpholino, thiomorpholino, N—C₁-C₇-alkyl-piperazino, or N-mono- or N,N-di-(C₁-C₇-alkyl)-amino-substituted or unsubstituted pyrrolidino; where as aryl phenyl or naphthyl, each of which is unsubstituted or substituted by one or more, e.g. up to three, substituents independently selected from the group consisting of C₁-C₇-alkyl, hydroxy-C₁-C₇-alkyl, C₁-C₇-alkoxy-C₁-C₇-alkyl, halo-C₁-C₇-alkyl, pyrrolidino-C₁-C₇-alkyl, piperidino-C₁-C₇-alkyl, morpholino-C₁-C₇-alkyl, thiomorpholino-C₁-C₇-alkyl, N—C₁-C₇-alkyl-piperazino-C₁-C₇-alkyl, N-mono- or N,N-di-(C₁-C₇-alkyl)-amino-substituted or unsubstituted pyrrolidino-C₁-C₇-alkyl, halo, especially fluoro, chloro or bromo, hydroxy, C₁-C₇-alkoxy, C₁-C₇-alkoxy-C₁-C₇-alkoxy, amino-C₁-C₇-alkoxy, N—C₁-C₇-alkanoylamino-C₁-C₇-alkoxy, carbamoyl-C₁-C₇-alkoxy, N-mono- or N,N-di-(C₁-C₇-alkyl)-carbamoyl-C₁-C₇-alkoxy, amino, C₁-C₇-alkanoylamino, C₁-C₇-alkanoyl, C₁-C₇-alkoxy-C₁-C₇-alkanoyl, carboxy, C₁-C₇-alkoxycarbonyl, carbamoyl, N-mono- or N,N-di-(C₁-C₇-alkyl and/or C₁-C₇-alkoxy-C₁-C₇-alkyl)-carbamoyl, pyrrolidinocarbonyl, piperidinocarbonyl, morpholinocarbonyl, thiomorpholinocarbonyl, N—C₁-C₇-alkyl-piperazinocarbonyl, N-mono- or N,N-di-(C₁-C₇-alkyl)-amino-substituted or unsubstituted pyrrolidino-C₁-C₇-alkyl, nitro, cyano, pyrrolidino, piperidino, morpholino, thiomorpholino, N—C₁-C₇-alkyl-piperazino, and N-mono- or N,N-di-(C₁-C₇-alkyl)-amino-substituted or unsubstituted pyrrolidino; is especially preferred;
in unsubstituted or substituted heterocyclyl is mentioned, heterocyclyl is a heterocyclic radical that is unsaturated, saturated or partially saturated in the bonding ring and is preferably a monocyclic or in a broader aspect of the invention poly-, e.g. bi- or tri-cyclic ring; has 3 to 24, more preferably 4 to 16 ring atoms; wherein at least in the ring bonding to the remaining part of the molecule of formula I one or more, preferably one to four, especially one or two carbon ring atoms are replaced by a heteroatom selected from the group consisting of nitrogen, oxygen and sulfur, the bonding ring preferably having 4 to 12, especially 5 to 7 ring atoms; heterocyclyl being present unsubstituted or substituted by one or more, especially 1 to 3, substituents independently selected from the group consisting of the substituents defined above under “substituted alkyl” or “substituted aryl”; especially being a heterocyclyl radical selected from the group consisting of oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, pyranyol, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, indolizinyl, isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, beta-carbolinyl, phenanthridinyl, acridinyl, perimidinyl, phenanthrolinyl, furazanyl, phenazinyl, phenothiazinyl, phenoxazinyl, chromenyl, isochromanyl and chromanyl, each of these radicals being unsubstituted or substituted by one to two radicals selected from the group consisting of lower alkyl, especially methyl or tertbutyl, lower alkoxy, especially methoxy, and halo, especially bromo or chloro;
unsubstituted or substituted cycloalkyl is mono- or polycyclic, more preferably monocyclic, C₃-C₁₆-cycloalkyl, especially C₃-C₁₀-cycloalkyl which may include one or more double and/or triple bonds, and is unsubstituted or substituted by one or more, e.g. one to three substituents preferably independently selected from those mentioned above as substituents for substituted alkyl or substituted aryl; where cyclopropyl, cyclobutyl, cyclopentyl, cyclohexyl or cycloheptyl are especially preferred;
n is, 1, 2 or 3, preferably 0 or 1, e.g. 0;
m is 0, 1, 2 or 3, preferably 0 or 1, e.g. 0;
p is 0, 1, 2 or 3, preferably 1 or 2, most preferably 1;
etherified or esterified hydroxy is hydroxy etherified by unsubstituted or substituted lower alkyl which is as defined above, and is more preferably lower-alkoxy, (lower-alkoxy)-lower alkoxy, phenoxy, naphthoxy, phenyl-lower alkoxy, such as benzyloxy, or naphthyl-lower alkoxy; or hydroxy esterified by an organic carbonic or sulfonic acid, e.g. lower alkanoyloxy, lower alkoxy-carbonyloxy, such as tert-butoxycarbonyloxy, phenyl-lower alkoxy-carbonyloxy, such as benzyloxycarbonyloxy, methylphenylsulfonyloxy or lower-alkylsulfonyloxy;
substituted mercapto is mercapto that is thioesterified with acyl as defined below, especially with lower alkanoyloxy; or preferably thioetherified with alkyl, aryl, heterocyclyl or cycloalkyl each of which is unsubstituted or substituted and is preferably as described above for the corresponding unsubstituted or substituted moieties, where unsubstituted or especially substituted C₁-C₇-alkylthio or unsubstituted or substituted arylthio with unsubstituted or substituted C₁-C₇-alkyl or aryl as just described for the corresponding moieties under etherified hydroxy are especially preferred;
acyl is preferably unsubstituted or substituted aryl-carbonyl or -sulfonyl, unsubstituted or substituted heterocyclylcarbonyl or -sulfonyl, unsubstituted or substituted cycloalkylcarbonyl or -sulfonyl, formyl or unsubstituted or substituted alkylcarbonyl or -sulfonyl, wherein unsubstituted or substituted aryl, unsubstituted or substituted heterocyclyl, unsubstituted or substituted cycloalkyl and unsubstituted or substituted alkyl are preferably as described above; preferably acyl is C₁-C7-alkanoyl, C₁-C₇-alkylsulfonyl or (unsubstituted or C₁-C₇-alkyl-substituted) phenylsulfonyl;
substituted amino is mono- or di-substituted amino, wherein amino is preferably substituted by one or two substituents selected from one acyl, especially C₁-C₇-alkanoyl, C₁-C₇-alkylsulfonyl or phenylsulfonyl wherein phenyl is unsubstituted or substituted by one to 3 C₁-C₇-alkyl groups, and one or two moieties selected from alkyl, aryl, heterocyclyl and cycloalkyl each of which is unsubstituted or substituted and is preferably as described above for the corresponding unsubstituted or substituted moieties; preferably substituted amino is C₁-C₇-alkanoylamino, mono- or di-[C₁-C₇-alkyl and/or C₁-C₇-alkoxy-C₁-C₇alkyl and/or (mono- or di-(C₁-C₇-alkyl)-amino-C₁-C₇-alkyl]-amino or mono- or di-(naphthyl- or phenyl-C₁-C₇-alkyl)-amino, or (where the binding nitrogen forms part of a ring) piperidino, morpholino, thiomorpholino, N—C₁-C₇-alkyl-piperazino, or N-mono- or N,N-di-(C₁-C₇-alkyl-substituted or unsubstituted pyrrolidino;
esterified carboxy is alkyloxycarbonyl, aryloxycarbonyl, heterocyclyloxycarbonyl or cyclo alkyloxycarbonyl, wherein alkyl, aryl, heterocyclyl and cycloalkyl are unsubstituted or substituted and the corresponding moieties and their substituents are preferably as described above; esterified carboxy is preferably C₁-C₇-alkoxycarbonyl, phenyl-C₁-C₇-alkoxycarbonyl, phenoxycarbonyl or naphthoxycarbonyl;
in mono- or disubstituted carbamoyl, the amino part is unsubstituted or substituted as described for substituted amino, but not with acyl as amino substituent, or is an N that forms part of an unsubstituted or substituted heterocyclyl ring, especially pyrrolidino, morpholino, thiomorpholino, piperazino or N—C₁-C₇-alkylpiperazino; preferred is mono- or di-(C₁-C₇-alkyl and/or C₁-C₇-alkoxy-C₁-C₇alkyl)-aminocarbonyl, mono- or di-(naphthyl- or phenyl-C₁-C₇-alkyl)-aminocarbonyl, pyrrolidinocarbonyl, morpholino-carbonyl, thiomorpholinocarbonyl, pyrrolidinocarbonyl, piperazinocarbonyl or N—C₁-C₇-alkylpiperazinocarbonyl;
esterified sulfo is alkyloxysulfonyl, aryloxysulfonyl, heterocyclyloxysulfonyl or cycloalkyloxysulfonyl, wherein alkyl, aryl, heterocyclyl and cycloalkyl are unsubstituted or substituted and the corresponding moieties and their substituents are preferably as described above, where C₁-C₇-alkoxysulfonyl, phenyl-C₁-C₇-alkoxysulfonyl, phenoxysulfonyl or naphthoxysulfonyl are especially preferred;
in N-mono- or N,N-disubstituted sulfamoyl, the amino part is unsubstituted or substituted as described for substituted amino, but preferably with no acyl as amino substituent; preferred is mono- or di-(C₁-C₇-alkyl and/or C₁-C₇-alkoxy-C₁-C₇alkyl)-aminosulfonyl or mono- or di (naphthyl- or phenyl-C₁-C₇-alkyl)-aminosulfonyl;
X₁ is CH, X₂ is N or CH and X₃ is CH;
and arylene is a bivalent aryl with an aryl ring system as defined above for aryl and heterocyclylene is a bivalent heterocyclyl with a heterocyclyl ring system as defined above for heterocyclyl;
or a pharmaceutically acceptable salt thereof.

3. A compound of the formula I according to claim 1, wherein
R1 is selected from the group consisting of amino, N-mono- or N,N-di-C₁-C₇-alkylamino, C₁-C₇-alkanoylamino, C₁-C₇-alkoxy-C₁-C₇-alkanoylamino, C₁-C₇-alkoxycarbonylamino, N-mono- or N,N-di-C₁-C₇-alkoxy-C₁-C₇alkylamino, N-mono- or N,N-di-(C₁-C₇-alkyl)-amino-C₁-C₇-alkylamino, optionally C₁-C₇-alkyl-substituted pyrrolidinyl-C₁-C₇-alkylamino, optionally C₁-C₇-alkyl-substituted morpholinyl-C₁-C₇-alkylamino, optionally C₁-C₇-alkyl-substituted piperazinyl-C₁-C₇-alkylamino and optionally C₁-C₇-alkyl-substituted piperidinyl-C₁-C₇-alkylamino;
R2 is C₁-C₇-alkyl or hydrogen;
n is 0 or 1;
m is 0 or 1;
p is 0, 1 or, 2;
each of R3 and R4, independently of the other, is hydrogen, C₁-C₇-alkyl, halo, hydroxy, C₁-C₇-alkoxy, nitro, amino, N-mono- or N,N-di-C₁-C₇-alkylamino, carboxy, C₁-C₇-alkoxycarbonyl, carbamoyl, N-mono- or N,N-di-C₁-C₇-alkyl-carbamoyl, sulfo, sulfamoyl or cyano;
R5 is C₁-C₇-alkyl, phenyl, furyl or C₃-C₈-cycloalkyl;
R6 is phenyl which is unsubstituted or substituted by up to three substituents independently selected from the group consisting of C₁-C₇-alkyl, halo-C₁-C₇-alkyl, C₁-C₇-alkoxy, C₁-C₇-alkylsulfonyl, C₁-C₇-alkoxy-C₁-C₇-alkoxy-C₁-C₇-alkoxy, C₁-C₇-alkoxycarbonyl, benzyloxy, cyano, N-mono- or N,N-di-(C₁-C₇-alkyl)-amino-C₁-C₇-alkyl, N—(N′-mono- or N′,N′-di-(C₁-C₇-alkyl)-amino-C₁-C₇-alkyl)-N—(C₁-C₇-alkyl)-amino-C₁-C₇-alkyl, pyrrolidino-C₁-C₇-alkyl, piperidino-C₁-C₇-alkyl, morpholino-C₁-C₇-alkyl, N—C₁-C₇-alkyl-piperazino-C₁-C₇-alkyl, N-mono or N,N-di-(C₁-C₇-alkyl)-amino-substituted pyrrolidino-C₁-C₇-alkyl, pyrrolidinocarbonyl, piperidino-carbonyl, morpholinocarbonyl, N—C₁-C₇-alkyl-piperazin-carbonyl, N-mono- or N,N-di-(C₁-C₇-alkyl)-amino-substituted pyrrolidinocarbonyl and halo;
X₁ and X₃ are CH and X₂ is N;
Y is imino (—NH—), thio (—S—) or oxy (—O—);
Ar is phenylene;
and one of Z₁ and Z₂ is N, the other is CH;
or a pharmaceutically acceptable salt thereof.

4. A compound of the formula I according to claim 1, wherein,
R1 is selected from the group consisting of amino, methylamino, (1-methyl-pyrrolidin-2-yl)-ethylamino, (pyrrolidin-1-yl)-ethylamino, (morpholin-4-yl)-ethylamino, (morpholin-4-yl)-propylamino, 2-(N,N-dimethyl-amino)-ethylamino, (4-methyl-piperazin-1-yl)-propylamino, (2-methyl-piperidin-1-yl)-propylamino, (1-methyl-piperidin-4-yl)-amino, (1-ethyl-pyrrolidin-2-yl)-methylamino, carbamic acid methylester and 2-methoxy acetamide;
R2 is hydrogen;
n is 0
m is 0 or 1;
p is 1;
R4 is hydrogen, methoxy or fluoro;
R5 is tert-butyl which is bound in position 5 of the pyrazolyl ring in formula I;
R6 is phenyl which is unsubstituted or substituted by a substituent selected from the group consisting of methyl, isopropyl, trifluoromethyl, methoxy, methyl-sulfonyl, methoxy-ethoxy-methoxy, methoxycarbonyl, benzyloxy, cyano, N,N-dimethylaminomethyl, N—(N′,N′-dimethylaminopropyl)-N-methyl-aminomethyl, morpholinomethyl, 4-methyl-piperazinomethyl, N,N-dimethyl-amino-pyrrolidino-methyl, morpholinocarbonyl, 4-methyl- or 4-isopropyl-piperazinocarbonyl, N,N-dimethyl-amino-pyrrolidino-carbonyl, fluoro, chloro and bromo;
X₁ and X₃ are CH and X₂ is N;
Y is oxy (—O—);
Ar is 1,4-phenylene;
Z₁ is CH;
Z₂ is N;
or a pharmaceutically acceptable salt thereof.

5. A compound of the formula I according to claim 1 selected from the following group of compounds:
1-[4-(6-amino-pyrimidin-4-yloxy)-phenyl-3-(5-tert-butyl-2-p-tolyl-2H-pyrazol-3-yl)-urea;
1-[(5-tert-butyl-2-p-tolyl-2H-pyrazol-3-yl)-3-[4-(6-methylamino-pyrimidin-4-yloxy)-phenyl]-urea;
1-[5-tert-butyl-2-(4-morpholin-4-ylmethyl-phenyl)-2H-pyrazol-3-yl]-3-[4-(6-methylamino-pyrimidin-4-yloxy)-phenyl]-urea;
1-[4-(6-amino-pyrimidin-4-yloxy)-phenyl]-3-[5-tert-butyl-2-(4-morpholin-4-ylmethyl-phenyl)-2H-pyrazol-3-yl]-urea;
1-[4-(6-amino-pyrimidin-4-yl-oxy)-phenyl]-3-(5-cyclopentyl-2-pyridin-4-yl-2H-pyrazol-3-yl)-urea;
1-[4-(6-amino-pyrimidin-4-yl-oxy)-phenyl]-3-(5-phenyl-2-p-tolyl-2H-pyrazol-3-yl)-urea;
1-[4-(6-amino-pyrimidin-4-yl-oxy)-phenyl]-3-(5-furan-2-yl-2-p-tolyl-2H-pyrazol-3-yl)-urea;
1-[5-tert-butyl-2-(4-fluoro-phenyl)-2H-pyrazol-3-yl]-3-(4-{6-[2-(1-methyl-pyrrolidin-2-yl)ethylamino]-pyrimidin-4-yloxy}-phenyl)-urea;
(6-{4-[3-(5-tertbutyl(4-methanesulfonyl-phenyl)-2H-pyrazol-3-yl)ureido]-phenoxy}-pyrimidin-4-yl)-carbamic acid methylester;
(6-{4-[3-(5-tert-butyl(4-methanesulfonyl-phenyl)-2H-pyrazol-3-yl)-ureido]phenoxy}-pyrimidin-4-yl]-2-methoxy acetamide;
1-(5-tert-butyl-2-m-tolyl-2H-pyrazol-3-yl)-3-[3-methoxy-4-(6-methylamino-pyrimidin-4-yloxy)-phenyl]-urea;
1-[4-(6-amino-pyrimidin-4-yloxy)-3-fluoro-phenyl]-3-[5-tert-butyl-2-(4-methoxy-phenyl]-2H-pyrazol-3-yl]-urea;
1-[4-(6-amino-pyrimidin-4-yloxy)-2-fluoro-phenyl]-3-[5-tert-butyl-2-(4-fluoro-phenyl]-2H-pyrazol-3-yl]-urea;
1-{5-tert-butyl-2-[3-(4-methyl-piperazine-1-carbonyl)-phenyl]-2H-pyrazol-3-yl}-3-[4-(6-methylamino-pyrimidin-4-yloxy)-phenyl]-urea;
1-[4-(6-amino-pyrimidin-4-yloxy)-phenyl]-3-{5-tert-butyl-2-[4-(morpholine-4-carbonyl)-phenyl]-2H-pyrazol-3-yl}-urea;
1-{5-tert-butyl-2-[4-(morpholine-4-carbonyl)-phenyl]-2H-pyrazol-3-yl}-3-[4-(6-methylamino-pyrimidin-4-yloxy)-phenyl]-urea
and compounds of the formula 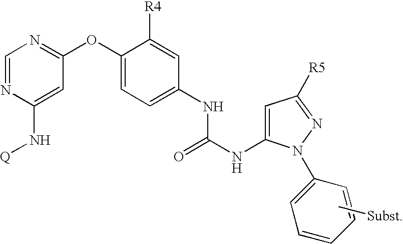
as represented in the following table:

| R4 | R5 | Subst. | Q |
|---|---|---|---|
| H | tert-butyl | 4-isopropyl | H |
| H | tert-butyl | | CH₃ |
| H | tert-butyl | 4-CF₃ | H |
| H | tert-butyl | | CH₃ |
| H | tert-butyl | 3-CH₃ | H |
| H | tert-butyl | | CH₃ |
| H | tert-butyl | 4-F | CH₃ |
| H | tert-butyl | 4-OCH₃ | H |
| H | tert-butyl | 4-SO₂CH₃ | H |
| H | tert-butyl | 4-SO₂CH₃ | CH₃ |
| H | tert-butyl | 4-OMEM | H |
| H | tert-butyl | 4-SO₂CH₃ | H |
| H | tert-butyl | 4-OBn | H |
| H | tert-butyl | 4-CN | H |
| H | cyclopentyl | 4-CH₃ | H |
| H | cyclopentyl | 4-OCH₃ | H |
| H | cyclopentyl | 4-CN | H |
| H | cyclopentyl | 4-SO₂CH₃ | H |
| H H | tert-butyl tert-butyl | 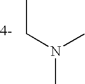 | H CH₃ |
| H | tert-butyl | 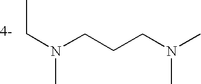 | CH₃ |
| H H | tert-butyl tert-butyl | 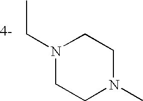 | H CH₃ |
| H H | tert-butyl tert-butyl | 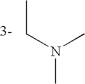 | H CH₃ |
| H H | tert-butyl tert-butyl | 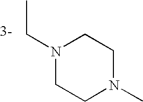 | H CH₃ |
| H H | tert-butyl tert-butyl | 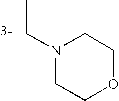 | H CH₃ |
| H H | tert-butyl tert-butyl | 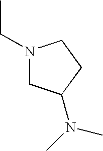 | H CH₃ |
| H | tert-butyl | 4-F | 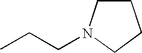 |
| H | tert-butyl | 4-F | 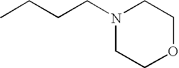 |
| H | tert-butyl | 4-F | 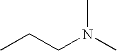 |
| H | tert-butyl | 4-F | 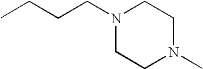 |
| H | tert-butyl | 4-F | 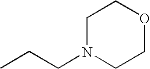 |
| H | tert-butyl | 4-F | 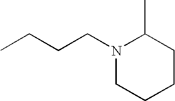 |
| H | tert-butyl | 4-F | 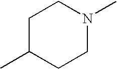 |
| H | tert-butyl | 4-F | 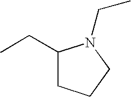 |
| OCH₃ | tert-butyl | 4-iso-propyl | CH₃ |
| OCH₃ | tert-butyl | 4-SO₂CH₃ | H |
| OCH₃ | tert-butyl | 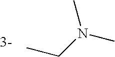 | CH₃ |
| OCH₃ | tert-butyl | 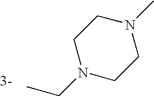 | CH₃ |
| OCH₃ | tert-butyl | 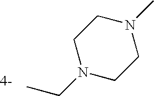 | CH₃ |
| OCH₃ | tert-butyl | 4-OCH₃ | H |
| OCH₃ | tert-butyl | 4-F | CH₃ |

or a pharmaceutically acceptable salt thereof.

6. A compound of the formula I, or a pharmaceutically acceptable salt thereof, according to claim 1 for use in the diagnostic or therapeutic treatment of a warm-blooded animal.

7. A compound of the formula I, or a pharmaceutically acceptable salt thereof, according to claim 1 in the treatment of a disease that depends on activity of a protein kinase, especially Tie-2 kinase.

8. The use of a compound of the formula I, or a pharmaceutically acceptable salt thereof, according to claim 1 for the manufacture of a pharmaceutical composition for the treatment of a disease that depends on activity of a protein kinase, especially Tie-2 kinase.

9. The use of a compound of the formula I, or a pharmaceutically acceptable salt thereof, according to claim 1 for the treatment of a disease that depends on activity of a protein kinase, especially Tie-2 kinase.

10. A pharmaceutical formulation, comprising a compound of the formula I, or a pharmaceutically acceptable salt thereof, according to claim 1 and at least one pharmaceutically acceptable carrier material.

11. A method of treatment a disease that depends on activity of a kinase, especially Tie-2 kinase, comprising administering to a warm-blooded animal, especially a human, in need of such treatment a pharmaceutically effective amount of a compound of the formula I, or a pharmaceutically acceptable salt thereof according to claim 1.

12. A process for the preparation of a compound of the formula I according claim 1, comprising reacting an isocyanate compound of the formula II, 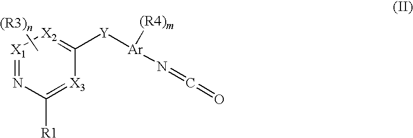
wherein R1, R3, R4, X₁, X₂, X₃, Ar, n and m are as defined for a compound of the formula I, with an amino compound of the formula III, 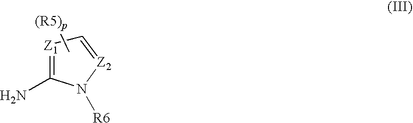
wherein R5, R6 and p are as defined for a compound of the formula I,
and, if desired, transforming an obtainable compound of formula I into a different compound of formula I, transforming a salt of an obtainable compound of formula I into the free compound or a different salt transforming an obtainable free compound of formula I into a salt thereof and/or separating an obtainable mixture of isomers of a compound of formula I into individual isomers;
where in any starting materials functional groups that shall not take part in the reaction may be present in protected form and protecting groups are removed to obtain a compound of the formula I.
